(19) United States
(12) Patent Application Publication
(10) Pub. No.: US 2025/0146060 A1
(43) Pub. Date: May 8, 2025

(54) ECHO AMPLIFICATION: A COMPREHENSIVE SYSTEM OF CHEMISTRY AND METHODS FOR AMPLIFICATION AND DETECTION OF SPECIFIC NUCLEIC ACID SEQUENCES

(71) Applicant: Cepheid, Sunnyvale, CA (US)

(72) Inventors: Glenn C. Johns, Palo Alto, CA (US); Carla Maria McDowell-Buchanan, Bothell, WA (US); Vera Baraznenok, Sunnyvale, CA (US)

(21) Appl. No.: 18/691,007

(22) PCT Filed: Sep. 15, 2022
(86) PCT No.: PCT/US2022/043702

§ 371 Date: Mar. 11, 2024

Related U.S. Application Data
(60) Provisional application No. 63/245,149, filed on Sep. 16, 2021.

(51) Int. Cl. C12Q 1/686; C12N 9/12; C12N 9/16; C12N 15/11; C12Q 1/6813; C12Q 1/6853; C12Q 1/6876

(52) U.S. Cl. CPC C12Q 1/686; C12N 9/1252; C12N 9/16; C12N 15/11; C12Q 1/6813; C12Q 1/6853; C12Q 1/6876; C12Y 207/07007

(57) ABSTRACT

Described herein is a comprehensive novel amplification system that can employ novel nucleic acid constructs as blocked primers, such constructs including a target-specific region, wherein the target-specific region comprises a target-specific cleavage domain, and a universal adapter sequence located 5′ of the target-specific region, wherein the universal adapter sequence comprises a non-natural nucleotide base. Also described is a fusion protein between a polymerase and an endonuclease, which is useful in this system, as well as related methods and kits.

### CROSS-REFERENCE TO RELATED APPLICATIONS

This application claims the benefit of U.S. provisional application No. 63/245,149, filed Sep. 16, 2021, which is hereby incorporated by reference in its entirety.

### STATEMENT AS TO RIGHTS TO DISCLOSURES MADE UNDER FEDERALLY SPONSORED RESEARCH AND DEVELOPMENT

Not applicable.

### FIELD

The methods and compositions described herein relate generally to the area of nucleic acid amplification.

### BACKGROUND

A wide variety of nucleic acid amplification methods are available, and many have been employed in the implementation of sensitive diagnostic assays based on nucleic acid detection. Polymerase chain reaction (PCR) remains the most widely used DNA amplification and quantitation method. Illustrative PCR-related methods and compositions are described in the following patents and publications:
U.S. Pat. No. 4,965,188 Process for Amplifying, Detecting, and/or Cloning Nucleic Acid Sequences Using a Thermostable Enzyme-PCR;
U.S. Pat. No. 5,011,769 Methods for Detecting Nucleic Acid Sequences-Cycling Probe Technology;
U.S. Pat. No. 5,066,584 Methods for Generating Single Stranded DNA by the Polymerase Chain Reaction—Asymmetric PCR;
U.S. Pat. No. 5,432,272 Method for Incorporating into DNA or RNA Oligonucleotide Using Nucleotides Bearing Heterocyclic Bases—Non-natural Bases;
U.S. Pat. No. 5,712,124 Strand Displacement Amplification-SDA Isothermal Amplification;
U.S. Pat. No. 5,994,056 Homogeneous Methods for Nucleic Acid Amplification and Detection-Real Time PCR;
U.S. Pat. No. 6,150,097 Nucleic Acid Detection Probes Having Non-FRET Fluorescence Quenching and Kits and Assays Including Such Probes-Molecular Beacons;
U.S. Pat. No. 6,174,670 B1 Monitoring Amplification of DNA During PCR—HRM Probes;
U.S. Pat. No. 6,410,278 B1 Process for Synthesizing Nucleic Acid—Loop-Mediated Isothermal Amplification (LAMP);
U.S. Pat. No. 7,270,981 B2 Recombinase Polymerase Amplification—RPA Isothermal Amplification;
U.S. Pat. No. 7,282,328 B2 Helicase Dependent Amplification of Nucleic Acids—HDA Isothermal Amplification;
U.S. Pat. No. 7,381,818 B2 Fluorescent Probes Containing 5′ Minor Groove Binder, Fluorophore and Quenching Moieties and Methods of Use Thereof-MGB Pleaides Probes;
U.S. Pat. No. 7,485,442 B2 Real-time Linear Detection Probes: Sensitive 5′-Minor Grove Binder-containing Probes for PCR Analysis-MGB Eclipse Probes;
U.S. Pat. No. 8,911,948 B2 RNase H-Based Assays Utilizing Modified RNA Monomers—RNase H2-dependent PCR (rhAmp PCR);
U.S. Pat. No. 9,689,031 B2 Nicking and Extension Amplification Reaction for the Exponential Amplification of Nucleic Acids-NEAR Isothermal Amplification;
US Patent Application 2019/0226015 A1 Improvements in or Relating to Nucleic Acid Amplification Processes—STAR Non-isothermal NEAR Amplification; and

Don et al. 1991. ‘Touchdown’ PCR to circumvent spurious priming during gene amplification. Nucleic Acids Research Vol. 19 No. 14: p. 4008.

These methods represent some of the greatest innovations in molecular biology over the past three decades, particularly as they relate to different methodologies for the amplification and real-time detection of multiple DNA or RNA analytes. While each has both strengths and limitations to varying degrees, the single largest limitation of the entire collection of art is their lack of coherence in a single amplification and detection system to provide all their collective attributes on a single clinical sample of interest.

Multidomain enzymes do occur in nature that carry multiple functions targeting enhancement of native characteristics. The art has sought to enhance the natural characteristics of polymerases where, for example, fusion of the sequence-nonspecific DNA binding helix-hairpin-helix domains of topoisomerase V to the catalytic domains of DNA polymerases is used to enhance DNA binding, improving processivity, thermal stability or salt tolerance.^{1,2} This has been demonstrated with enzymes such as the Stoffel fragment of Taq DNA polymerase, Pfu DNA polymerase, ϕ29 DNA polymerase and Bst DNA polymerase. Heterologous DNA binding proteins have also been fused to DNA polymerases. Fusion of Sso7d from Sulfolobus solfataricus to Pyrococcus furiosus (Pfu polymerase) and Taq DNA polymerase improved processivity.³ Polymerase processivity enhancement and improved tolerance to PCR inhibitors was demonstrated through fusion of Taq Stoffel DNA polymerase with the DNA-binding domain of Pyrococcus furiosus ligase.⁴

Peptide linkers are routinely employed to fuse polypeptides to form fusion proteins. Serine rich linkers provide increased solubility and improved resistance to proteolysis.^{5,7} Helical linkers are inserted between domains of recombinant chimeras that aid in higher levels of expression and reduced interdomain interactions.^{6,7}
1. Pavlov, A., et. al. Biochemistry 2012, 51, 2032-2043.
2. Pavlov, A., et. al. PNAS, 2002, 99, 13510-13515.
3. Wang, Y., et. al. Nucleic Acids Res. 2004, 32(3), 1197-1207.
4. Spibida, M., et. al. Applied Microbiology and Biotechnology, 2018, 102(2), 713-721.
5. U.S. Pat. No. 5,525,491 Serine-rich peptide linkers.
6. U.S. Pat. No. 7,943,733 Spacers to increase the expression of recombinant fusion proteins.
7. Chen, X., et. al. Advance Drug Delivery Reviews 65(2013) 1357-1369.

Further descriptions of polymerases can be found in the following patents.
U.S. Pat. No. 9,023,633 Chimeric DNA polymerases
U.S. Pat. No. 8,404,808 Phage phi29 DNA polymerase chimera
U.S. Pat. No. 7,148,049 Thermostable or thermoactive DNA polymerase molecules with attenuated 3′-5′ exonuclease activity
U.S. Pat. No. 8,883,454 DNA polymerase fusions and uses thereof
U.S. Pat. No. 5,814,506 Over Expression and purification of a truncated thermostable DNA polymerase by protein fusion
U.S. Pat. No. 9,434,988 RNase H based assays utilizing modified RNA monomers

### SUMMARY

Various embodiments contemplated herein may include, but need not be limited to, one or more of the following:

Embodiment 1: A nucleic acid construct comprising: a target-specific region; and a universal adapter sequence located 5′ of the target-specific region, wherein the universal adapter sequence comprises a non-natural nucleotide base.

Embodiment 2: The nucleic acid construct of embodiment 1, wherein the nucleic acid construct additionally comprises a terminal 3′ cap.

Embodiment 3: The nucleic acid construct of embodiment 1 or embodiment 2, wherein the target-specific region comprises a target-specific cleavage domain.

Embodiment 4: The nucleic acid construct of embodiment 3, wherein the target-specific cleavage domain comprises a ribonucleotide.

Embodiment 5: The nucleic acid construct of any one of embodiments 1-4, wherein the non-natural nucleotide base is a ribonucleotide.

Embodiment 6: The nucleic acid construct of any one of embodiments 1-4, where in the non-natural nucleotide base is a deoxyribonucleotide.

Embodiment 7: The nucleic acid construct of any one of embodiments 1-4, wherein the non-natural nucleotide base is selected from the group consisting of xanthosine, isoguanosine, deoxyxanthosine, deoxyisoguanosine, isocytosine, deoxyisocytosine, 6-amino-5-nitro-3-(1′-beta-D-2′-ribofuranosyl)-2(1H)-pyridone, 6-amino-5-nitro-3-(1′-beta-D-2′-deoxyribofuranosyl)-2(1H)-pyridone, 2-amino-8-(1′-beta-D-2′-ribofuranosyl)-imidazo[1,2-a]-1,3,5-triazin-4(8H)-one, and 2-amino-8-(1′-beta-D-2′-deoxyribofuranosyl)-imidazo[1,2-a]-1,3,5-triazin-4(8H)-one.

Embodiment 8: The nucleic acid construct of embodiment 7, wherein the non-natural nucleotide base is selected from the group consisting of 6-amino-5-nitro-3-(1′-beta-D-2′-ribofuranosyl)-2(1H)-pyridone or a 6-amino-5-nitro-3-(1′-beta-D-2′-deoxyribofuranosyl)-2(1H)-pyridone, 2-amino-8-(1′-beta-D-2′-ribofuranosyl)-imidazo[1,2-a]-1,3,5-triazin-4(8H)-one, and 2-amino-8-(1′-beta-D-2′-deoxyribofuranosyl)-imidazo[1,2-a]-1,3,5-triazin-4(8H)-one.

Embodiment 9: The nucleic acid construct of embodiment 7, wherein the non-natural nucleotide base is a 2-amino-8-(1′-beta-D-2′-ribofuranosyl)-imidazo[1,2-a]-1,3,5-triazin-4(8H)-one or a 2-amino-8-(1′-beta-D-2′-deoxyribofuranosyl)-imidazo[1,2-a]-1,3,5-triazin-4(8H)-one.

Embodiment 10: A fusion protein comprising: a polymerase, or a fragment thereof, and an endonuclease, or a fragment thereof.

Embodiment 11: The fusion protein of embodiment 10, wherein the polymerase is a Bst DNA polymerase, or a fragment thereof.

Embodiment 12: The fusion protein of embodiment 11, wherein the polymerase is the large fragment of the Bst DNA polymerase.

Embodiment 13: The fusion protein of embodiment 11, wherein the polymerase is a Bst 2.0 DNA polymerase, or a fragment thereof.

Embodiment 14: The fusion protein of any one of embodiments 10-13, wherein the endonuclease is an endoribonuclease.

Embodiment 15: The fusion protein of embodiment 14, wherein the endoribonuclease is an RNase H2, or a fragment thereof.

Embodiment 16: A primer pair comprising: a forward primer and a reverse primer, the forward and reverse primers each comprising the nucleic acid construct of any one of embodiments 1-9.

Embodiment 17: The primer pair of embodiment 16, wherein the forward primer additionally comprises a terminal 3′ cap.

Embodiment 18: The primer pair of embodiment 16 or embodiment 17, wherein the reverse primer additionally comprises a terminal 3′ cap.

Embodiment 19: The primer pair of any one of embodiments 16-18, wherein the non-natural nucleotide base of forward primer is the same as the non-natural nucleotide base of reverse primer.

Embodiment 20: The primer pair of any one of embodiments 16-18, wherein the non-natural nucleotide base of forward primer is different from the non-natural nucleotide base of reverse primer.

Embodiment 21: The primer pair of any one of embodiments 16-20, wherein the non-natural nucleotide base of forward primer is a ribonucleotide and the non-natural nucleotide base of reverse primer is a ribonucleotide.

Embodiment 22: The primer pair of embodiment 17, wherein the non-natural nucleotide base of forward primer is a ribonucleotide and the non-natural nucleotide base of reverse primer is a deoxyribonucleotide.

Embodiment 23: A method of detecting a target nucleic acid sequence, the method comprising:
a) providing a reaction mixture comprising
   i) the primer pair of any one of embodiments 16-22;
   ii) a sample nucleic acid that may or may not comprise the target sequence;
   iii) a cleaving activity; and
   iv) a polymerase activity;
b) hybridizing a first primer of the primer pair to the target nucleic acid sequence, if present, to form a first double-stranded substrate comprising the target nucleic acid sequence and a first target-specific cleavage domain;
c) cleaving the hybridized first primer with a first cleaving activity at a point within or adjacent to a first target-specific cleavage domain;
d:) extending the primer with the polymerase activity to form a first template;
e) hybridizing a second primer of the primer pair to the first template to form a second double-stranded substrate comprising the target nucleic acid sequence and a second target-specific cleavage domain;
f) cleaving the hybridized second primer with a second cleaving activity at a point within or adjacent to the second cleavage domain;
g) extending the primer with the polymerase activity to form a first amplicon comprising, on a first strand, a first non-natural nucleotide base derived from the first primer;
h) cleaving the first strand at or adjacent to the first non-natural nucleotide with a third cleavage activity, creating a first nick with a 3′ hydroxyl group; and
i) extending, from the 3′ hydroxyl group, with the polymerase activity, displacing the portion of the first stand 3′ of the first nick, optionally wherein the first, second, and third cleavage activities are the same.

Embodiment 24: The method of embodiment 22, wherein terminal 3′ end of each primer comprises a terminal 3′ cap.

### BRIEF DESCRIPTION OF THE DRAWINGS

FIGS. 1 and 2: Schematic drawings showing various elements of illustrative embodiments of the chimeric primers described herein. FIG. 1 shows looped (“hairpin”) primers, and FIG. 2 shows linear primers. The architecture of the chimeric primers includes two main segments. As shown for both the hairpin and linear versions, the universal adapter sequences (1) can have optional non-natural DNA and ribobases that are non-palindromic to the target sequence and designed to have a TM greater than the reaction temperature. The target-specific sequence (4) (not including the terminal 3′ cap) has a Tm greater than the reaction temperature.

FIG. 3 is a schematic illustrating how a single-stranded DNA template for amplification can be produced. In the case of DNA, genomic DNA suffers routine environmental damage from any number of causes. Genomic DNA damage can also result from ultrasonication. In both cases, nicks are introduced into the genomic DNA, providing a point from which a strand displacing polymerase can act to synthesize a new strand of DNA, while displacing the portion of DNA 3′ of the nick. In the case of RNA, standard reverse transcription can be carried out to synthesize a new strand of DNA. The action of the strand displacing polymerase or reverse transcriptase thus provides a single strand of DNA to facilitate an amplification reaction.

FIG. 4 is a schematic depicting illustrative embodiments that begin with hybridization of either a hairpin or linear primer that is complementary to the target nucleotide sequence. Both primer versions are unblocked on the 3′-end by an endonuclease (RNase H2) leaving a free 3′-hydroxyl available for polymerase extension (this “unblocking” or “3′-activation” step is illustrated only for the hairpin primer but occurs in the same manner with the linear primer). In one embodiment, a first hairpin primer hybridizes to a single-stranded DNA template, is unblocked (3′-activated), and is extended by a strand displacing polymerase. A second hairpin primer may then invade the end of the helix, undergo hybridization, 3′-activation, and extension, allowing the strand displacing polymerase to displace the newly formed strand. In some embodiments, shown here for the linear primer, an additional primer that hybridizes to a non-target sequence 5′ of the target sequence can be used to enhance strand displacement. This additional primer can be a cleavable (e.g., 3′-activatable) strand displacement initiation primer that is not complementary to the target sequence, and after hybridization, 3′-activation, and subsequent extension by a strand displacing polymerase, this initiation primer displaces the extension product of the linear primer which includes the universal adapter sequence of the linear primer, as well as the target-specific sequence of interest (the extension product of the initiation primer does not contain the universal adapter sequence).

FIG. 5 shows a schematic illustrating hybridization of a new primer (here, a “reverse,” linear primer) to the extension product of the (here, “forward”) linear primer of FIG. 4. Hybridization of the reverse, linear primer to a target sequence in the extension product of the forward, linear primer, creates a DNA duplex that is recognized by RNase H2, which cleaves the 3′-end of the reverse, linear primer, leaving a free 3′-end available for extension (i.e., 3′activation). Translation of the polymerase through the 5′-end of the universal adapter sequence (derived from the forward, linear primer) generates a double-stranded nucleic acid segment (“first adapter duplex”) including a mixture of natural and non-natural bases and excluding the target-specific sequence. Formation of the first adapter duplex generates a recognition site for RNase H2 activity. In one embodiment, cleavage 5′ of the non-natural ribobase in the first adapter duplex leaves a free 3′-OH for extension by a strand-displacing polymerase that displaces a 3′ portion of the extension product of the forward, linear primer, enabling translation of the polymerase through the 5′-end of the of the reverse, linear primer forming a second adapter duplex including a mixture of natural and non-natural bases, an RNase H2 recognition site, and excluding target-specific sequence. To generate the RNase H2 recognition site, a non-natural ribobase can be provided in the reaction so that the strand displacing polymerase inserts a non-natural ribobase to generate the RNase H2 recognition site in the second adapter complex. This cleavage and strand displacement process generates a double-stranded DNA (dsDNA) species containing the target-specific sequence with adapter duplexes at both the 5′ and 3′ ends, which is termed the “full amplification duplex.” Cleavage by RNase H2 (shown in the first adapter duplex) and subsequent extension and displacement of the 3′ portion of the cleaved strand generates a single-stranded DNA (ssDNA) species truncated on the 5′-end with a non-natural ribobase and a 3′-end containing a mixture of natural and non-natural bases. This ssDNA species represents a linear amplification product. These displaced ssDNA linear amplification products are generated in both the forward or reverse directions (as shown) and are available for probe hybridization. In the illustrative embodiment shown here, the probe is a linear probe containing a single natural ribobase which is located in between a quencher (Q) and fluorescent label (F). Hybridization of the probe to the linear amplification product generates an RNase H2 recognition site. Cleavage by RNase H2, 5′ of the ribobase in the probe, releases a 5′ fragment with a permanently unquenched fluorophore.

FIG. 6 is a schematic showing another function for a linear amplification product produced as described in FIG. 5 in an illustrative embodiment. Here, a displaced linear amplification product with the target sequence flanked at the 5′-end with a non-natural ribobase and at the 3′ end with an adapter sequence. A new reverse primer binds the displaced linear amplification product with complementary sequences to the target sequence and the adapter sequence. Cleavage at the RNase H2 recognition site produced upon this hybridization, followed by extension, generates a reverse hemi-amplification duplex (“reverse” because the duplex results from extension from the reverse primer, and “hemi” because the amplification duplex does not include all the elements of the full amplification duplex; here, the full forward adapter duplex is missing. RNase H2 cleavage of the hemi-amplification duplex, followed by extension and strand displacement, generates a ssDNA species including a target-specific sequence flanked on the 3′ and 5′ ends with a non-natural deoxybase and non-natural ribobase respectively. This ssDNA species represents a truncated version of the linear amplification product, but one that can still be detected by a probe.

FIGS. 7 and 8 are schematics representing hybridization of either the forward or reverse primers to a truncated linear amplification product containing the target-specific sequence. This hybridization event creates an RNase H2 recognition site; RNase H2 cleaves 5′ of the native ribobase base unblocking the primers allowing for extension (ultimately, in both directions) by a strand displacing polymerase. FIG. 7 shows a forward primer hybridizing, resulting in the formation of forward hemi-amplification duplex. In the same reaction, reverse primers will also hybridize to the truncated linear amplification product, forming reverse hemi-amplification duplexes, as shown in FIG. 8. Reverse or forward hemi-amplification duplexes are formed in either order resulting in two species where the target sequence is flanked on one end with an adapter complex with an RNase H2 recognition site and the other end with non-natural 3′ deoxynucleotide and a 5′ non-natural ribonucleotide (on separate strands and paired with one another). RNase H2 cleavage in the adapter duplex generates a further truncated linear amplification product that is displaced as extension occurs across from the template strand, which also regenerates the forward and reverse hemi-amplification duplexes (see FIG. 7, showing regeneration of the forward hemi-amplification duplex, and FIG. 8, showing regeneration of the reverse hemi-amplification duplex). The displaced further truncated linear amplification product is available for detection by the cycling probe (used in this illustrative embodiment; though those of skill in the art readily appreciate that any number of hybridization probes can be employed for detection of the target gene sequence).

FIGS. 9 and 10 illustrate a detection pathway for a Single Nucleotide Polymorphism (SNP). In this embodiment, the hairpin or linear primers (see FIG. 9) are capped on the 3′-end with a sequence including a ribobase that is correctly paired with the SNP of interest. The SNP-driven activation of the primer occurs through RNase H2 cleavage and removal of the terminal 3′ cap, allowing for primer extension by a strand displacing polymerase in both the forward and reverse directions leading to a full amplification duplex (see FIG. 10). Detection of the SNP is facilitated using a probe with the same sense as one of the primers. In some embodiments, the probe is a cycling probe with the native ribobase being an exact complement to the SNP in the target sequence. Amplification and detection proceed as outlined in FIGS. 1-8.

FIG. 11 shows an illustrative summary of the products formed through various isothermal amplification pathways that can exist when the processes outlined in FIGS. 1-8 are carried out. Circular arrows depict the linear amplification pathways, while the horizontal arrows shown are the exponential pathways. Amplification of each strand can be controlled independently in the forward direction (solid arrows) and the reverse direction (dashed arrows) through controlling the ratios of different non-natural and natural NTPs and dNTPs, primer mixtures of non-natural ribobases and deoxynucleoside bases, or primer biasing. In one embodiment, utilization of a cycling probe allows continued detection of the forward strand (as shown). In some embodiments, the cycling probe detection is in the reverse direction. Continued probe detection, in either direction, as the amplification pathways progress through the linear and exponential phases is accomplished with the strand that is more abundant.

FIG. 12 shows additional pathways whereby the minimal amplification duplex can be restored to hemi-amplification duplexes through additional priming and extension events. Further priming and extension events lead to hemi-amplification duplexes being restored to full amplification duplexes.

FIG. 13 shows results of visual endpoint monitoring of reaction mixtures subjected to isothermal amplification using linear primers, as described in FIG. 2 above in the presence or absence of 20,000 copies of Neisseria gonorrhoeae genomic DNA at 70° C. for 25 minutes. Lanes 2 and 3 show the full-amplification duplexes, the hemi-amplification duplexes, and the minimal amplification duplexes at endpoint. Lanes 4 and 5 are no template controls (NTC), and Lane 1 shows molecular weight markers.

FIGS. 14A and 14B show the results of fluorescence monitoring (FIG. 14A) and visual endpoint monitoring (FIG. 14B) of reaction mixtures subjected to isothermal amplification using linear primers in the presence or absence of Streptococcus pyogenes Group A gDNA over a 10-fold dilution series. Reactions progressed at 70° C. over the course of 25 minutes. FIG. 14B, Lane 1 shows molecular weight markers, Lanes 2-5 are endpoint reactions, and Lane 6 is an NTC.

FIGS. 15A and 15B show the results of fluorescence monitoring of isothermal amplification using linear primers over a 10-fold dilution series (FIG. 15A) and visual endpoint monitoring at different KCl concentrations (FIG. 15B). Reaction mixtures produced in the presence or absence of gDNA from Bacillus subtilis Strain 168. Reactions progressed at 70° C. for 20 minutes. FIG. 15B, Lane 1-10K copies/reaction at 40 mM KCl, Lane 2-10K copies/reaction at 70 mM KCl, Lane 3-NTC reaction at 40 mM KCl, and Lane 4-molecular weight markers.

FIGS. 16A and 16B show the results of fluorescence monitoring of isothermal amplification using linear primers over a 10-fold dilution series (FIG. 16A) and visual endpoint monitoring of 20K copies/reaction (FIG. 16B). Reaction mixtures produced in the presence or absence of gDNA from Chlamydia trachomatis Serovar D. Reactions progressed at 70° C. for 20 minutes. FIG. 16B, Lane 1—molecular weight markers, Lanes 2-7—endpoint products from 20K genomic copies, and Lanes 5-7-NTC reactions.

FIG. 17 is a schematic showing a chimera of two enzymes harboring two disparate functions that are fused with a peptide linker. The first enzyme (Activity A) acts upon a unique substrate generating an intermediate product that serves as a substrate for the active site of the second enzyme (Activity B). Juxtaposing enzyme activities (Activity A+B), through covalent linkages, allows for bridging consecutive reactions resulting in the desired product.

FIGS. 18A and 18B are 4-20% gradient SDS-PAGE gels showing IPTG induction, and the soluble expression of chimeras constructed from the fusion of Bst LF DNA polymerase and RNase H2. For both of FIGS. 18A and 18B, Lane 1—molecular weight standards. Lanes 2, 4, 6, and 8—non-induced cells, and Lanes 3, 5, 7, and 9—soluble expression fractions after ITPG induction. FIG. 18A shows the results for chimeras having SEQ ID NOs: 4-7, and FIG. 18B shows the results for chimeras having SEQ ID NOs: 8-11.

FIG. 19 is a 4-20% gradient SDS-PAGE gel showing results for the purified chimeras having SEQ ID NOs: 4, 8, 10, and 6 (from left to right) and molecular weight standards (Lanes 1 and lane to the left of Lane 6).

FIG. 20 shows results of maximum rates generated from fluorescence monitoring of RNase H2 cleavage activity (“Activity A”). Cleavage reactions were performed at 65° C. for 15 minutes using SEQ ID NO:14 as the activity substrate at 300 nM. Assays were run in 20 μL reactions using 0.1 mU/μL RNase H2 and 5 mU/μL for Bst LF and SEQ ID NO:4, SEQ ID NO:6, SEQ ID NO:8, and SEQ ID NO: 10. The activity substrate, SEQ ID NO:14, is 3′-end labelled with FAM (6-carboxyfluorescein) terminated by a C3 spacer. The 5′-end was labelled with Cepheid's CDQ13R quencher. The substrate contained a single ribonucleotide (lower case). RNase H2 cleavage results in a short FAM fragment that dissociates and fluoresces allowing real-time monitoring. Bars show average of four replicate measurements, and error bars show standard deviation.

FIG. 21 shows relative maximum polymerase extension rates (“Activity B”) for Bst LF and fusion enzymes (SEQ ID NO:4, SEQ ID NO:6, SEQ ID NO: 8, and SEQ ID NO:10). Assays were run in 20 μL reactions using 5 mU/μL enzyme at 65° C. Fluorescence measurements were collected for 18 minutes. The reporter substrate (SEQ ID NO:15) is a fluorescence-quenched double hairpin. Bold T is dT-Dabcyl, and bold C is a Cepheid cytosine 44 analog. Extension of the 3′-terminus by a polymerase opens the hairpin structure increasing the distance between the fluorophore-quencher pair and a positive signal is detected at 520 nm. Bars show average of four replicate measurements, and error bars show standard deviation.

FIG. 22 shows the maximum rates and the induced response halfway between the minimum and maximum over the reaction duration (t₅₀) for concerted endonuclease and polymerase activities (“Activity A+B”) for the fusion enzymes (SEQ ID NO:4, SEQ ID NO:6, SEQ ID NO:8, and SEQ ID NO:10). Assays were run in 20 μL reactions using 5 mU/μL enzyme at 65° C. and monitored for 18 minutes. The activity substrate (SEQ ID NO:16) is a fluorescence-quenched double hairpin. Bold T is dT-Dabcyl, the 3′-end is blocked with a single RNA base (lower case) embedded within the stem region, DNA bases 3′ of the ribobase, and terminated with a C3 spacer. Presence of RNase H2 activity cleaves and activates the 3′-end for extension by the polymerase domain. Extension by a polymerase increases the distance between the fluorophore-quencher pair and a positive signal is detected at 520 nm. Light grey bars are t₅₀ in minutes. Dark grey bars are maximum rates in percentage per minute. Fluorescence signals plateaued to similar endpoints and were normalized to derive maximum rates. Bars show average of four replicate measurements, and error bars show standard deviation.

FIG. 23 shows the comparison of maximum rates for concerted endonuclease and polymerase activities (“Activity A+B”) for the one-enzyme system (SEQ ID NO:6), and the two-enzyme system (Bst LF and RNase H2) on an annealed linear substrate system. Assays were run in 20 μL reactions using 5 mU/μL enzyme at 65° C. and monitored for 18 minutes. The amount of enzyme used in each reaction are based on the number of moles as determined by A280 measurements and associated extinction coefficients. The template sequence is 5′-labelled with a Cepheid quencher 5′-CDQ13R-GCGTAGCTGACTGCAGCTGCA GCGACGGCGTCACTGATTGTGCACAGAGGCGCCTCGAGCGC-3′ (SEQ ID NO: 17), the blocked primer sequence is 5′-G (+C) G (+C) TCGAG GCG cCT CTG-C3-3′ (SEQ ID NO:18), where + indicates LNA base and lowercase indicates native ribobase, the reporter sequence is 5′-GCTGCAGCTGCAGTCAGCTACGC (FAM)-C3-3′ (SEQ ID NO:19). Bars show average of four replicate measurements, and error bars show standard deviation.

### DETAILED DESCRIPTION

### Definitions

Terms used in the claims and specification are defined as set forth below unless otherwise specified.

The term “nucleic acid” refers to a nucleotide polymer, and unless otherwise limited, includes analogs of natural nucleotides that can function in a similar manner (e.g., hybridize) to naturally occurring nucleotides.

The term nucleic acid includes any form of DNA or RNA, including, for example, genomic DNA; complementary DNA (cDNA), which is a DNA representation of mRNA, usually obtained by reverse transcription of messenger RNA (mRNA) or by amplification; DNA molecules produced synthetically or by amplification; mRNA; and non-coding RNA.

The term nucleic acid encompasses double- or triple-stranded nucleic acid complexes, as well as single-stranded molecules. In double- or triple-stranded nucleic acid complexes, the nucleic acid strands need not be coextensive (i.e., a double-stranded nucleic acid need not be double-stranded along the entire length of both strands).

The term nucleic acid also encompasses any modifications thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, sugar-phosphate backbone modifications, unusual base pairing combinations such as the isobases isocytidine and isoguanidine, and the like.

More particularly, in some embodiments, nucleic acids can include xanthosine, isoguanosine, deoxyxanthosine, deoxyisoguanosine, isocytosine, deoxyisocytosine, 6-amino-5-nitro-3-(1′-beta-D-2′-ribofuranosyl)-2(1H)-pyridone, 6-amino-5-nitro-3-(1′-beta-D-2′-deoxyribofuranosyl)-2(1H)-pyridone, 2-amino-8-(1′-beta-D-2′-ribofuranosyl)-imidazo[1,2-a]-1,3,5-triazin-4(8H)-one, 2-amino-8-(1′-beta-D-2′-deoxyribofuranosyl)-imidazo[1,2-a]-1,3,5-triazin-4(8H)-one, polydeoxyribonucleotides (containing 2-deoxy-D-ribose), polyribonucleotides (containing D-ribose), and any other type of nucleic acid that is an N- or C-glycoside of a purine or pyrimidine base, as well as other polymers containing nonnucleotidic backbones, for example, polyamide (e.g., peptide nucleic acids (PNAs)) and polymorpholino polymers (see, e.g., Summerton and Weller (1997) “Morpholino Antisense Oligomers: Design, Preparation, and Properties,” Antisense & Nucleic Acid Drug Dev. 7:1817-195; Okamoto et al. (20020) “Development of electrochemically gene-analyzing method using DNA-modified electrodes,” Nucleic Acids Res. Supplement No. 2:171-172), and other synthetic sequence-specific nucleic acid polymers providing that the polymers contain nucleobases in a configuration which allows for base pairing and base stacking, such as is found in DNA and RNA. The term nucleic acid also encompasses locked nucleic acids (LNAs), which are described in U.S. Pat. Nos. 6,794,499, 6,670,461, 6,262,490, and 6,770,748, which are incorporated herein by reference in their entirety for their disclosure of LNAs.

The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

Individual nucleotides in DNA or RNA are described herein as “bases” generally; as “deoxyribonucleotides” or “deoxybases” for nucleotides in DNA; or as “ribonucleotides” or “ribobases in RNA. Nucleotides can be natural or non-natural. “Natural” nucleotides are those known to occur in nature as of the original filing date of the present disclosure. “Non-natural” nucleotides are those not known to occur in nature as of the original filing date of the present disclosure.

As used herein, the term “complementary” refers to the capacity for precise pairing between two nucleotides; i.e., if a nucleotide at a given position of a nucleic acid is capable of hydrogen bonding with a nucleotide of another nucleic acid to form a canonical base pair, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. The degree of complementarity between nucleic acid strands has significant effects on the efficiency and strength of hybridization between nucleic acid strands.

“Specific hybridization” refers to the binding of a nucleic acid to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

In some embodiments, hybridizations are carried out under stringent hybridization conditions. The phrase “stringent hybridization conditions” generally refers to a temperature in a range from about 5° C. to about 20° C. or 25° C. below than the melting temperature (Tₘ) for a specific sequence at a defined ionic strength and pH. As used herein, the Tₘ is the temperature at which a population of double-stranded nucleic acid molecules becomes half-dissociated into single strands. Methods for calculating the Tₘ of nucleic acids are well known in the art (see, e.g., Berger and Kimmel (1987) METHODS IN ENZYMOLOGY, VOL. 152: GUIDE TO MOLECULAR CLONING TECHNIQUES, San Diego: Academic Press, Inc. and Sambrook et al. (1989) MOLECULAR CLONING: A LABORATORY MANUAL, 2ND ED., VOLS. 1-3, Cold Spring Harbor Laboratory), both incorporated herein by reference for their descriptions of stringent hybridization conditions). As indicated by standard references, a simple estimate of the Tₘ value may be calculated by the equation: Tₘ=81.5+0.41 (% G+C), when a nucleic acid is in aqueous solution at 1 M NaCl (see, e.g., Anderson and Young, Quantitative Filter Hybridization in NUCLEIC ACID HYBRIDIZATION (1985)). The melting temperature of a hybrid (and thus the conditions for stringent hybridization) is affected by various factors such as the length and nature (DNA, RNA, base composition) of the primer or probe and nature of the target nucleic acid (DNA, RNA, base composition, present in solution or immobilized, and the like), as well as the concentration of salts and other components (e.g., the presence or absence of formamide, dextran sulfate, polyethylene glycol). The effects of these factors are well known and are discussed in standard references in the art. Illustrative stringent conditions suitable for achieving specific hybridization of most sequences are: a temperature of at least about 60° C. and a salt concentration of about 0.2 molar at pH7. Tₘ calculation for oligonucleotide sequences based on nearest-neighbors thermodynamics can carried out as described in “A unified view of polymer, dumbbell, and oligonucleotide DNA nearest-neighbor thermodynamics” John SantaLucia, Jr., PNAS Feb. 17, 1998 vol. 95 no. 4 1460-1465 (which is incorporated by reference herein for this description).

The term “oligonucleotide” is used to refer to a nucleic acid that is relatively short, generally shorter than 200 nucleotides, more particularly, shorter than 100 nucleotides, most particularly, shorter than 50 nucleotides. Typically, oligonucleotides are single-stranded DNA molecules.

The term “primer” refers to an oligonucleotide that is capable of hybridizing (also termed “annealing”) with a nucleic acid and serving as an initiation site for nucleotide (RNA or DNA) polymerization under appropriate conditions (i.e., in the presence of four different nucleoside triphosphates and an agent for polymerization, such as DNA or RNA polymerase or reverse transcriptase) in an appropriate buffer and at a suitable temperature. The appropriate length of a primer depends on the intended use of the primer, but primers are typically at least 7 nucleotides long and, in some embodiments, range from 10 to 30 nucleotides, or, in some embodiments, from 10 to 60 nucleotides, in length. In some embodiments, primers can be, e.g., 15 to 50 nucleotides long. Short primer molecules generally require cooler temperatures to form sufficiently stable hybrid complexes with the template. A primer need not reflect the exact sequence of the template but must be sufficiently complementary to hybridize with a template. A primer generally contains a sequence that is complementary to a sequence (termed the “target sequence”) present in sample nucleic acids. The sequence in the primer is said to be “target-specific.”

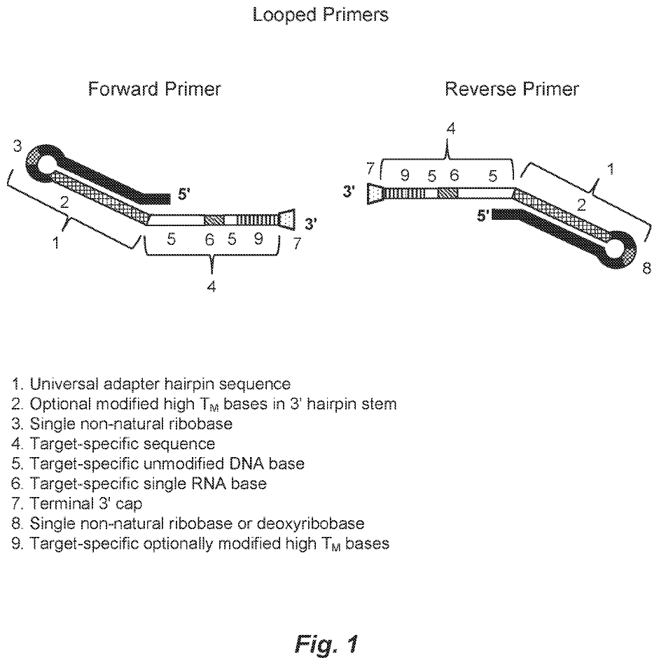

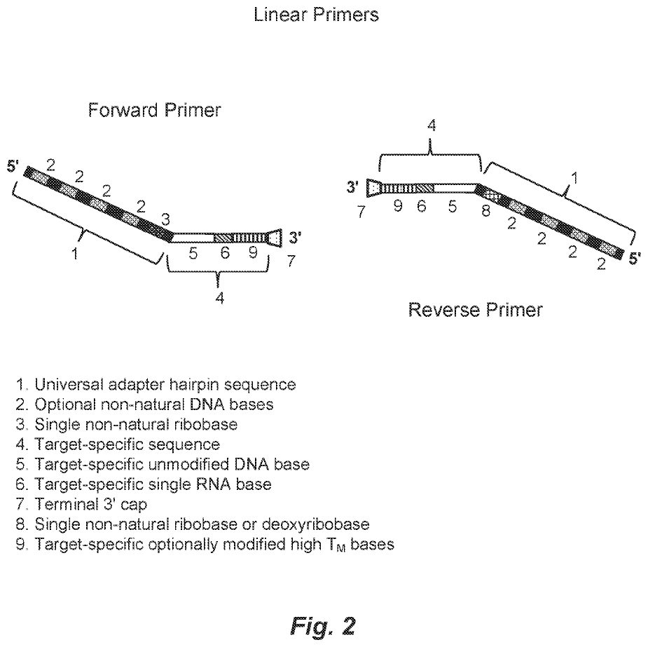

A “chimeric” primer is one that contains a plurality of different functional elements (i.e., more than simply a target-specific sequence). Illustrative chimeric primers are shown in FIGS. 1 and 2. Chimeric primers are employed in the methods described herein, where they are sometimes referred to simply as “primers,” for ease of discussion.

With reference to an element of a primer, the term “cap” refers to a structure (typically, at the terminus of the primer) that cannot be extended by a polymerase. The primer bearing a cap, e.g., a 3′ terminal cap is said to be “blocked.” A cap can be removed or deactivated to allow the primer to prime the production of an extension produced by a polymerase, and the process is referred to herein as “activation.”

A primer is said to anneal to another nucleic acid if the primer, or a portion thereof, hybridizes to a nucleotide sequence within the nucleic acid. The statement that a primer hybridizes to a particular nucleotide sequence is not intended to imply that the primer hybridizes either completely or exclusively to that nucleotide sequence. For example, in some embodiments, amplification primers used herein are said to “anneal to” or be “specific for” a nucleotide sequence.” This description encompasses primers that anneal wholly to the nucleotide sequence, as well as primers that anneal partially to the nucleotide sequence.

The term “primer pair” refers to a set of primers including a 5′ “upstream primer” or “forward primer” that hybridizes with the complement of the 5′ end of the DNA sequence to be amplified and a 3′ “downstream primer” or “reverse primer” that hybridizes with the 3′ end of the sequence to be amplified. As will be recognized by those of skill in the art, the terms “upstream” and “downstream” or “forward” and “reverse” are not intended to be limiting, but rather provide illustrative orientations in some embodiments.

A “probe” is a nucleic acid capable of binding to a target nucleic acid of complementary sequence through one or more types of chemical bonds, generally through complementary base pairing, usually through hydrogen bond formation, thus forming a duplex structure. The probe can be labeled with a detectable label to permit facile detection of the probe, particularly once the probe has hybridized to its complementary target. Alternatively, however, the probe may be unlabeled, but may be detectable by specific binding with a ligand that is labeled, either directly or indirectly. Probes can vary significantly in size. Generally, probes are at least 7 to 15 nucleotides in length. Other probes are at least 20, 30, or 40 nucleotides long. Still other probes are somewhat longer, being at least 50, 60, 70, 80, or 90 nucleotides long. Yet other probes are longer still, and are at least 100, 150, 200 or more nucleotides long. Probes can also be of any length that is within any range bounded by any of the above values (e.g., 15-20 nucleotides in length).

The primer or probe can be perfectly complementary to the target nucleotide sequence or can be less than perfectly complementary. In some embodiments, the primer has at least 65% identity to the complement of the target nucleotide sequence over a sequence of at least 7 nucleotides, more typically over a sequence in the range of 10-30 nucleotides, and, in some embodiments, over a sequence of at least 14-25 nucleotides, and, in some embodiments, has at least 75% identity, at least 85% identity, at least 90% identity, or at least 95%, 96%, 97%, 98%, or 99% identity. It will be understood that certain bases (e.g., the 3′ base of a primer) are generally desirably perfectly complementary to corresponding bases of the target nucleotide sequence. Primer and probes typically anneal to the target sequence under stringent hybridization conditions.

As used herein with reference to a portion of a primer or a nucleotide sequence within the primer, the term “specific for” a nucleic acid, refers to a primer or nucleotide sequence that can specifically anneal to the target nucleic acid under suitable annealing conditions.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially.

Illustrative means for performing an amplifying step include PCR, nucleic acid strand-based amplification (NASBA), two-step multiplexed amplifications, rolling circle amplification (RCA), and the like, including multiplex versions and combinations thereof, for example but not limited to, OLA/PCR, PCR/OLA, LDR/PCR, PCR/PCR/LDR, PCR/LDR, LCR/PCR, PCR/LCR (also known as combined chain reaction—CCR), helicase-dependent amplification (HDA), and the like. Descriptions of such techniques can be found in, among other sources, Ausubel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1): 41-7, U.S. Pat. Nos. 6,027,998; 6,605,451, Barany et al., PCT Publication No. WO 97/31256; Wenz et al., PCT Publication No. WO 01/112579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27: e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13 (2): 294-307, and Landegren et al., Science 241:1077-80 (1988), Demidov, V., Expert Rev Mol Diagn. 2002 November; 2(6): 542-8, Cook et al., J Microbiol Methods. 2003 May; 53(2): 165-74, Schweitzer et al., Curr Opin Biotechnol. 2001 February; 12(1): 21-7, U.S. Pat. Nos. 5,830,711, 6,027,889, 5,686,243, PCT Publication No. WO0056927A3, and PCT Publication No. WO9803673A1.

As used herein, an “amplicon” refers to a product of amplification. As used herein, an amplicon is usually, but need not be, double-stranded. The nature of an amplicon (double-stranded versus single-stranded) is apparent from the context in which this term is used.

A “multiplex amplification reaction” is one in which two or more nucleic acids distinguishable by sequence are amplified simultaneously.

The term “qPCR” is used herein to refer to quantitative real-time polymerase chain reaction (PCR), which is also known as “real-time PCR” or “kinetic polymerase chain reaction;” all terms refer to PCR with real-time signal detection.

A “reagent” refers broadly to any agent used in a reaction, other than the analyte (e.g., nucleic acid being analyzed). Illustrative reagents for a nucleic acid amplification reaction include, but are not limited to, buffer, metal ions, polymerase, reverse transcriptase, primers, template nucleic acid, nucleotides, labels, dyes, nucleases, dNTPs, and the like. Reagents for enzyme reactions include, for example, substrates, cofactors, buffer, metal ions, inhibitors, and activators.

The term “label,” as used herein, refers to any atom or molecule that can be used to provide a detectable and/or quantifiable signal. In particular, the label can be attached, directly or indirectly, to a nucleic acid or protein. Suitable labels that can be attached to probes include, but are not limited to, radioisotopes, fluorophores, chromophores, mass labels, electron dense particles, magnetic particles, spin labels, molecules that emit chemiluminescence, electrochemically active molecules, enzymes, cofactors, and enzyme substrates.

The term “dye,” as used herein, generally refers to any organic or inorganic molecule that absorbs electromagnetic radiation.

The naturally occurring bases adenine, thymine, uracil, guanine, and cytosine, which make up DNA and RNA, are described herein as “unmodified bases” or “unmodified forms.”

The term “modified base” is used herein to refer to a base that is not a canonical, naturally occurring base (e.g., adenine, cytosine, guanine, thymine, or uracil). Examples of modified bases are 2-thiothymine and 2-aminoadenine.

The term “modified high TM base” refers to a modified base that has a higher TM, when paired with a complementary nucleotide. One or more modified high TM bases can be included in an oligonucleotide, or portion thereof, to increase the TM of the oligonucleotide or portion.

Nucleotides comprising modified bases are referred to herein as “modified nucleotides.”

A DNA polymerase is said to be “stable” at a particular temperature if it provides a satisfactory extension rate in a nucleic acid amplification reaction.

The term “cycling probe” that can be cleaved by an enzyme after annealing to a target nucleic acid sequence, wherein such cleavage releases an intact target nucleic acid. A cycling probe enables a target nucleic acid to anneal to many molecules of the probe, thereby amplifying any signal associated with the probe.

The term “universal adapter sequence” is used herein to refer to a nucleotide sequence that is, or becomes, linked to a target nucleotide sequence. The universal adapter sequence is universal in the sense that, in a multiplex amplification reaction, amplicons produced from different targets will acquire the same adapter sequence. An adapter sequence typically facilitates amplification and/or downstream processing or analysis.

### INTRODUCTION

It is highly advantageous and desirable to be able to investigate with a single test a clinical, environmental, food, or other biological sample for the presence of specific nucleic acids for a myriad of reasons from infection to cancer to microbial contamination and so many more. In this regard, there are many potentially beneficial developments over the last 30 years, but they have been spread across many different chemistries and applications that are in many ways fundamentally incompatible-different temperature conditions such as temperature cycling, ramping, or low/medium/high isothermal temperatures, different buffer and pH conditions, radically different enzymes both in function and performance, and dramatically different approaches to probe designs and signal detection.

The present disclosure provides an entire system of amplification and detection chemistries and methods that can specifically detect virtually any DNA or RNA sequence, such as might be found in a pathogen, human genetic condition, RNA expression, cell-free DNA, or cancer cell for example. This assay occurs at a single temperature, or possibly at a slowly ramping or cycling temperature provided it is within the active range of the enzymes and oligos, to enable extremely fast (<10 min amplification), sensitive (<10 genome copies/reaction), and specific (capable of SNP discrimination) detection of a nucleic acid sequence of interest. Further, because of the enzymes and chemistry involved, this assay system is more robust and resistant to many common clinical sample inhibitors that often affect other molecular assays. This allows for the use of much simpler and faster sample preparation methods, thus allowing even greater time savings in total assay time. The enzymes used include a newly developed fusion enzyme that provides both a specific endonuclease function and a DNA-dependent DNA polymerase with strong strand displacement activity. These joint features enable the described reaction mechanism to rapidly amplify a specific DNA sequence when present, while at the same time preventing most all misamplification by mismatched sequences, primer-dimers, or other artifacts. Because of the specific endonuclease, all primers are initially blocked at the 3′ end and are only activated in the presence of the specific sequence of interest. This in turn enables a much higher level of multiplexing that typically has not been accomplished in other amplification (especially isothermal) systems.

For the detection of specific RNA sequences, a thermostable reverse transcriptase with RNase H activity can be included along with an unblocked cDNA synthesis primer at very low concentration. Alternatively, this can also be accomplished by either mutating the DNA-dependent DNA polymerase or altering its performance with reaction buffer excipients such as divalent manganese ions to induce additional RNA-dependent DNA polymerase activity as well. For double-stranded DNA targets, accessible single-stranded DNA for target amplification can be easily generated by the DNA polymerase with strong strand displacement activity extending from any nicks or gaps in the genomic DNA. In addition to other methods for introducing nicks into genomic DNA, sonication can provide for both efficient cell lysis and dsDNA nicking to provide accessible DNA for amplification. The primers can contain a number of different features in addition to the blocked 3′ end, including a target-specific (gene-specific) region with a ribobase for activating the primer when in contact with the target sequence, optionally a 3′ end with stabilizing high TM motif that can help facilitate strand invasion by the primer to generate additional amplicons, optionally a 5′ stabilizing hairpin in one embodiment that contains a second ribobase protected in a loop region that becomes exposed and active only after extension from the opposing primer displaces the hairpin. An alternative embodiment contains linear primers with a universal adapter sequence on the 5′ end that contains a unique sequence including non-natural DNA bases and a single non-natural ribobase. These protected ribobases are themselves comprised of two different xenobiotic non-natural base analogs which—in combination with their uniquely complementary non-natural base analogs—direct the incorporation of new ribobases into the replicated amplicon, thus allowing independent linear amplification of each strand even after all primers are consumed.

Because of special bases incorporated into the primer loops in one embodiment, the composition and concentration of the corresponding non-natural base nucleotide and deoxynucleotide triphosphates in the reaction can be controlled specifically and independently, which allows for the adjustment of asymmetric amplification in three different ways. In one approach, the ability to adjust linear amplification by adjusting NTP/dNTP ratios of one of the special non-natural nucleotides without having to greatly reduce one of the primer concentrations allows quick and efficient exponential amplification to proceed, while still producing excess of one strand to facilitate better probe binding and signal detection. Second, the primers can be provided in a mixed composition in which the non-natural base in the loop structure can be provided in the reaction as either an RNA ribobase or as a DNA base, whereby the ratio of the two primer variants determines the asymmetry of strand amplification. Third, the more conventional approach of skewing the forward and reverse primer concentrations also works well. The first and second approaches work well for maintaining efficient amplification kinetics because both primers are maintained at optimal concentrations, while the second and third approaches work well with high-level multiplexing as they can be adjusted individually for each amplicon.

Furthermore, the method can entail the use of any number of different types of hybridization probes, with a preferred embodiment utilizing linear probes with a ribobase towards the 5′ end for signal detection. While the probe will fluoresce when bound to its complementary sequence, the RNase H2 activity present in the reaction will also cleave the ribobase only when bound. The resulting short fragments will dissociate in solution leaving the fluorophore permanently unquenched and allowing a new probe molecule to bind to the same target sequence in what is known as Cycling Probe Technology (CPT). As the entire reaction can be isothermal, this cycling probe activity will proceed continuously, and greatly accelerate the detection of a positive fluorescent signal. Alternative hybridization probe designs such as Molecular Beacons or Pleaides probes will also work well, while each has their own benefits and limitations. Other embodiments include the use of an apurinic/apyrimidinic (AP) site in the probe to utilize Endonuclease III or IV.

Additional embodiments provide for the ability to detect and differentiate SNP's in a complex matrix background by targeting the target-specific (gene-specific) ribobase to the SNP location and moving the probe to the junction of the target-specific (gene-specific) region of the primer and the SNP-specific primer adapter sequence. Rare allele amplification and detection can also occur with this system by merely leaving the primer variant matching the dominant allele out of the reaction chemistry. Thus, the amplification of the gene will only occur if the rare allele is present to enable the RNase H2 activation of the primers.

Collectively, the amplification and detection system offer a comprehensive approach to provide all the needed and desired performance characteristics of high sensitivity, high specificity, rapid time to result, resistance to inhibition, and support for high-level multiplexing and even SNP detection and differentiation.

The polymerase art listed above in the Background demonstrates the notable advancements of protein engineering as it relates to enhancing naturally occurring functions of polymerases. These advancements have contributed to improvements of reagents used in molecular detection over a range of amplification techniques such as PCR, isothermal amplification, RCA, and SDA applications. The approach of fusing proteins with various DNA binding proteins or DNA-binding motifs yields built-in characteristics that improve the processivity, thermal stability, salt tolerance and improved tolerance to amplification inhibitors resulting from the sample type or chemical reagents used in the lysis reaction.

Multiple decades have passed with many polymerase improvements being made through domain swapping, active site mutations or the addition of DNA binding segments while leaving an uncharted landscape of fusing proteins with highly disparate functions. The current design space is limited in two key areas: 1) other fusions do not mix disparate functions such as polymerase and endonuclease, and 2) other systems which can incorporate a polymerase and an endonuclease as two separate enzymes do not have the same benefits that a one-enzyme system has. This disclosure is intended to address this gap in the field of polymerase engineering approaches for enzymes used in molecular diagnostics. Described herein are chimeric fusion enzymes that contain disparate functions that are not naturally occurring. An illustrative embodiment pairs the functionality of an endoribonuclease (RNase H2) with that of a DNA dependent DNA polymerase (Bst Large Fragment). The chimera provides the ability to excise a ribonucleotide base from an RNA-DNA hybrid and then extend a nascent DNA strand from the resultant free 3′-OH.

Also described is a one-enzyme system that is constructed such that the intermediate product formed from the first reaction, which forms a substrate for the second enzymatic reaction, is not lost to the surrounding bulk solution and competing reactions. This disclosure provides a fused protein scaffold that leads to an accelerated product formation versus a two-enzyme system.

Some embodiments include the use of various nucleases (for example SSIV, SSIII, Endo IV, CRISPR, Cas9 etc.) fused to other replicative polymerases (Magma, Pyrophage 3173, Vent, Phusion, 9° N, Pfu or Phi 29). It is also envisioned that the DNA dependent polymerase retains enough nascent reverse transcriptase activity such that the addition of a separate RT enzyme can not be required for the invention on RNA targets. In some embodiments, RNA dependent polymerases (RdRPs) can be used as fusion candidates since many viral polymerases possess associated domains that contain endonuclease or methyltransferase domains.

Described herein, is the fusion of two enzymes through flexible or rigid linkages without altering the individual characteristics of the enzymatic domains. Also described is the use of native peptide bonds that are part of the existing system (for example an amino acid sequence that exists as part of one of the natural sequences of either fusion protein sequence). In illustrative embodiments, natural peptides or chemical linkers, of varying lengths, form structural motifs and impart added flexibility or rigidity between the fused domains. The polymerase enzyme is not restricted to a particular DNA or RNA dependent polymerase. The endonuclease is not limited to a certain endoribonuclease. In fact, a variety of DNA or RNA dependent polymerases and endoribonucleases are considered with a variety of chimeric orientations and gene linkages. The engineered chimeras can be recombinantly generated from a variety of expression systems.

Production of a single chimera has a tremendous manufacturing advantage with respect to overall production time, manufacturing and quality control costs, and an overall reduction in development costs to bring a single product to commercial scale.

### Echo Amplification System

The current disclosure describes a complete amplification and detection system for the accurate and rapid detection of nearly any DNA or RNA molecular sequence. It is named Echo Amplification after the seminal characteristic of a cleavable site located in each primer—including an RNA ribobase in an illustrative embodiment—that is only activated after the complementary strand is fully synthesized, and only then initiates a second return of the strand synthesis along the nascent complementary strand, evocative of an echo reverberation off of a wall or terminus. The comprehensive inclusion of many different aspects of primer design, enzyme development, inclusion of natural and non-natural nucleotides, and fluorescent probe detection in an isothermal environment (in an illustrative embodiment) all come together to form a powerful system to deliver significant improvements in multiple facets of nucleic acid testing. This design process began by taking a systems-level approach to designing the amplification and detection chemistry. Various considerations taken into account during the design process are:

Rapid total assay time from initial sample to final answer, particularly for negative results.
Reducing the need and time for complex sample preparation steps.
Ability to function at a single isothermal reaction temperature to minimize time and energy demands of changing reaction temperatures, as well as removing physical checkpoints delaying reaction progression.
Use of novel chimeric enzymes which have disparate nuclease and polymerase functions fused together to reduce reaction time of separate sequential enzyme complex formations.
Designing multiple pathways for exponential amplification to proceed allows for more rapid amplification of target sequence.
Designing both asymmetric and independent linear amplification pathways to accelerate availability of single-stranded target DNA sequence for earlier detection.
Development of specific hybridization probes that function in real-time with additional fluorescence signal generation and acceleration utilizing cycling probe technology.

Design for excellent performance in both analytical and clinical sensitivity and specificity of target nucleic acid sequence.
Mismatch and non-specific amplification products are greatly reduced by utilizing blocked primers that when properly designed, only activate when hybridized to exactly complementary sequences.
Independent forward primer, reverse primer, and hybridization probe provide molecular specificity.
Numerous strains or sequence variants can be included for enhanced clinical sensitivity with multiple primers or probes in any single channel because of the blocked primers preventing misamplification.

High-level multiplexing, typically a challenge for isothermal amplification methods, is enabled by utilizing blocked primers and probes.
Many different primers can be included at moderate to even high concentrations because they are blocked and prevented from non-specific interactions such as primer-dimers and misamplifications that typically limit higher levels of multiplexing.
Reaction components such as primers, probes, enzymes, and nucleotide triphosphates are only consumed for the reactions that are present for a specific target sequence and are not consumed in off-target reactions.

To accomplish these design goals, it became clear early in the design process that it would be advantageous to have multiple features incorporated into the primer designs, as shown in FIGS. 1 and 2. In some embodiments, primers remain inactive unless and until they are hybridized to a specific target sequence. In some embodiments, primers incorporate universal adapters that then direct linear amplification of each strand independently. The linear amplification of each strand can then act as the template for the opposing primer, thus triggering an exponential amplification cycle. In some embodiments, each primer encodes the information signal to initiate a first-strand synthesis, and then also a return synthesis on subsequent rounds of amplification. One of shortest and simplest methods for achieving this is to include a ribonucleotide in the primer that when bound to the exact matching DNA sequence (i.e., when there is an exact match between the ribonucleotide and deoxynucleotide complement), then becomes the substrate for the excision repair pathway of RNase H2. After excision, RNase H2 leaves a free 3′ hydroxyl group on the primer fragment that was 5′ of the ribobase, which can then be extended by a DNA-dependent DNA polymerase with strand displacement activity. Though not required, with the use of a chimeric fusion enzyme that has both functions in different domains of the same enzyme, this process can proceed with both steps in rapid succession. Then, there can also be a second ribobase, a non-natural ribobase as depicted in FIG. 1, that is initially held in an inactive state, for example, by locating this second, non-natural ribobase unpaired in a small loop structure at the 5′ end of the primer. This second, non-natural ribobase in the loop structure is further protected because it is not composed of a naturally occurring base, so it is unlikely to ever come across its exact complement in a sample by chance. In some embodiments the primer incorporates a DNA sequence on the 5′ end that is not known to exist in any organism that might be present in a clinical sample, and thus that region would not bind to form an active site for cleavage even without a loop structure. This effect can be further enhanced by incorporating other non-natural DNA bases in this region to further differentiate the sequence from any naturally occurring nucleic acid sequences, as shown in FIG. 2. While the structures of the forward and reverse primers are conceptually similar, in the first embodiment each has a different ribobase in the loop region that is formed from different non-natural xenobiotic bases that do not have any complementarity to the standard A, C, G, T or U bases. Instead, they have their own base pair, with the complement nucleotide triphosphate provided in the reaction mixture. This design allows for the separate, independent control of forward and reverse strand amplification, minimizing wasted side reactions of cleaving just the very 3′ end of the amplicon off. Further, it also allows for the adjustment of the rate of ongoing linear amplification of each strand independently as well, through the control of nucleotide triphosphates versus deoxynucleotide triphosphates for the specific non-natural bases.

If used, the hairpin structure itself typically has a high enough TM to be stable at the reaction temperature and the combined length of the loop, hairpin stem, and unpaired end that are all on the 5′ side of the non-natural ribobase is preferably long enough to allow the combined operation of RNase H2 and DNA polymerase, if the loop should happen to be opened up by a strand displacing polymerase extending a hybridized complementary strand to the end of the primer. Typically, this is at least 8 to 12 nucleotides (e.g., 8, 9, 10, 11, or 12) for RNase H2 and comparable for Bst DNA polymerase. In other embodiments, the ribobases could be substituted with another method for triggering activation and a second activation, for example, by inclusion of an apurinic/apyrimidinic site and the enzymatic activity of a thermostable Endonuclease IV. In these alternative embodiments, linear amplification would occur with the inclusion of a primer in such cases. The strength of the hairpin would preferably have a TM just above the reaction temperature—nominally 65° C. but can be variable below the enzyme denaturation temperature—to remain in the hairpin configuration during the reaction and protect the internal non-natural ribobase. However, the hairpin TM would preferably be close to the reaction temperature to allow for the unfolding of the hairpin against its complementary sequence when bound to reaction intermediates in the exponential amplification phase. If any modified bases are needed to elevate the TM of this hairpin, they can be located on the 3′ side of the loop in order to allow for unfolding of the hairpin when the primer hybridizes to its fully complementary sequence, which will not contain any modified high TM bases as the complement will be synthesized by the DNA polymerase. While the use of modified high TM bases is not required, their use could allow the hairpin to be fewer base pairs in length than would otherwise be needed and thus allow for a shorter primer and hence a shorter amplicon overall. Additionally, the very 5′ end of the primer is preferably not part of the complementary palindromic sequence that forms the hairpin structure to prevent self-priming in subsequent rounds of amplification. While the 5′ end is naturally prevented from further extension if the strand should happen to fold onto itself, the complementary sequence formed by extension from the opposing primer would then also have a self-complementary sequence that could fold back onto itself in a hairpin, but would have a free unblocked 3′ hydroxyl capable of strand extension back across the nascent DNA, which would remove the DNA from any further amplification or detection, barring strand invasion.

Overall, the target-specific (gene-specific) region of the primer contains four different components, all of which are 3′ of the universal adapter region (FIG. 1 and FIG. 2). On the 5′ side of the target-specific (gene-specific) region is a section of at least 8-10 nucleotides which are unmodified and ideally an exact complement to the target sequence. This length allows for the RNase H2 enzyme to bind, recognize, and cleave a single ribobase that also has an exact match to the target sequence. Following this ribobase, there are an additional 2 to 4 unmodified nucleotides completing the region for RNase H2 recognition and potential cleavage. While modifications are possible in this region if desired, they are preferably limited to only those which do not interfere with RNase H2 or DNA polymerase activity. The 3′ side of this region is available to incorporate a stretch of modified high TM nucleotides that would confer additional specificity and stability if desired and would typically be from 4 to 12 nucleotides in length, though this stretch could be longer or shorter and still function appropriately. These high TM modified bases could be any number of potential bases that have been developed for increasing the TM of a particular DNA sequence and include for example locked nucleic acids (LNA's), 2,6-diaminopurine, 5-Methyl deoxycytidine, 5-hydroxybutynl-2′-deoxyuridine, 8-aza-7deazaguanosine, or other modified bases with similar characteristics. The benefits of these modified bases are that they can keep the primer length relatively short while still retaining good stability at the reaction temperature. These primers are then capped at the very 3′ end by any number of methods that are known for blocking the 3′ end of oligonucleotides to prevent polymerase extension, such as dideoxy ribose sugars, inverted bases attached in the reverse configuration with a 3′ to 3′ linkage, or a C3 spacer.

In some embodiments the high TM modified bases provide a method for additionally increasing the total amplification rate through a higher-than-natural base TM-mediated strand invasion. These modified bases are capable of stabilizing double-stranded DNA: modified DNA structure that is thermodynamically favored over their natural, unmodified counterparts consisting of only double-stranded DNA. Incorporating modified bases next to the 3′ end of the primer sequence allows for the invasion of the 3′ end of a new primer as the pairing of the high TM primer end is ultimately thermodynamically favorable over the same number of natural DNA base pairs. This strand invasion can then initiate a new round of amplification. This allows minimal amplification duplexes to be restored to partial hemi-amplification duplexes, and partial amplification duplexes to be restored to full amplification duplexes. It should be pointed out clearly that no strand invasion is necessary for exponential amplification to occur. Rather, strand invasion is part of a preferred embodiment that enables additional amplification pathways to continue to provide new target sequences accessible for amplification and detection, further decreasing the total assay time.

In some embodiments for Single Nucleotide Polymorphism (SNP) detection, the SNP can be placed at the exact location of the ribobase, wherein a single mismatch will not be cleaved by RNase H2, and no amplification could occur. Alternate primers with different SNP allele variants could all be included in a multiplex reaction, where the specific sequence of the universal region would comprise different sequences to tag alternate SNPs. Hybridization probes that are specific to the complement of the 3′ end of the opposite strand from the SNP-specific primer would then indicate which SNPs is present. For example, a SNP reaction where the forward strand was amplified in excess to the reverse strand, the reverse primer would then be the one with the SNP discriminating ribobase, and the probe would be specific to the junction of the 3′ gene and hairpin of the reverse primer, where the probe was the same sense as the reverse primer and only recognize the amplified complement to the reverse primer of that specific SNP.

Regarding enzymes used in the method described herein, one illustrative component would be a DNA-dependent DNA polymerase with strong strand displacing activity. In a particular embodiment, this activity is provided by a chimeric fusion enzyme that contains the large fragment of Bst DNA polymerase from Bacillus stearothermophilus. This fusion enzyme lacks any 5′ to 3′ exonuclease activity but does have an active RNase H2 endoribonuclease domain from Pyrococcus abyssi. Other embodiments can work with fusions between other DNA polymerases from other bacteria, archaea, or phages such as Bsu, Therminator, Phi29, or the Klenow fragment (exo-) and other endonucleases such as Apurinic/Apyrimidinic Endonuclease I, Endonuclease IV, or Endonuclease V. For the other nuclease approaches, the appropriate substrate would preferably be incorporated into the primers at the appropriate locations, such as the inclusion of an apurinic/apyrimidinic (AP) site in place of the ribobases present. The use of primers with AP sites would still allow the exponential amplification of target sequence through primer-directed amplification of each strand, but this approach would lack the additional linear amplification from the incorporation of specific ribobases by the polymerase on the nascent strand complementary to the primers. Consequently, these alternatives would likely be slower in the total reaction time, and thus would be less preferred embodiments.

In addition to the use of a chimeric fusion enzyme as described above, this amplification reaction mechanism works equally well with two separate enzymes—a polymerase such as Bst DNA polymerase and an endonuclease such as RNase H2—both supplied to the reaction. One aspect of this approach is that the fragments of the primers that are on the 5′ side of either nuclease target site, e.g. the ribobases, should typically have a sufficiently high TM such that the fragment remains bound to the target sequence after the action of the nuclease enzyme, and until the polymerase enzyme finds, binds, and extends the newly created 3′ hydroxyl. By having the two enzyme functions fused together, the kinetics of the overall reaction can be much faster and can thus enable the polymerase to immediately bind onto and extend the newly created hydroxyl, potentially even if the remaining 5′ fragment would not have a sufficient T_{M} to remain independently. This allows for the primer sequences to be shorter than would be optimal for the use of separate enzymes.

For the detection of RNA sequences such as RNA viruses, human mRNA, ribosomal RNA, or other RNA structures of interest, the RNA is typically reverse translated into a cDNA copy that can then be amplified for detection. This can be accomplished in several ways. In the preferred approach, additional cDNA synthesis primers are included that are unblocked at the 3′ end but will not contribute significantly to misamplification products as they are included at an exceptionally low concentration. A reverse transcriptase enzyme with RNase H+activity creates the cDNA first strand and hydrolyzes the RNA strand as it is copied, leaving a single-stranded cDNA for amplification. Examples of reverse transcriptases include RTx, HIV-1 RT, Omniscript, and mutants of Moloney-Murine Leukemia Virus (M-MLV) with enhanced thermostability and retaining RNase H activity. Reverse transcriptase enzymes that lack RNase H activity can equally be supplemented with a separate RNase H1 enzyme as well. Alternative embodiments include modifying the DNA polymerase to have at least limited RNA-dependent DNA polymerase activity either through mutation, e.g. Bst 3.0 polymerase, or through the supplement of reaction additives known to induce this effect in some wild-type DNA polymerases, for example the presence of divalent manganese ions with Bst polymerase. Indeed, both Bst DNA polymerase and RNase H2 have limited but measurable activity on RNA-RNA cleavage and RNA-dependent DNA polymerase, allowing the initial cDNA synthesis to occur without additional components at all. However, this rate of reaction initiation is expected to be much slower than the preferred approach.

Further, regarding enzymes, isothermal reaction conditions allow for the incorporation of cycling probe technology but utilizing a much more refined approach than the original approach using RNA probes and RNase H for cleavage. Here, the probe is included with a cleavable linkage that is recognized only when properly bound to the matching target sequence. In one embodiment, an endonuclease such as Endonuclease III would recognize the AP site in the middle of the probe when bound to target and cleave the AP site out of the bound probe. The fragment of the probe that lies on the 5′ side of the cleavage is left with a 3′-α, β-unsaturated aldehyde group that is not suitable for extension by DNA polymerase. Each of the two fragments on their own have a T_{M} much lower than the reaction temperature, and thus after cleavage the two fragments, quickly dissociate into solution, freeing up the target sequence to bind another intact probe and repeat the process. Once cleaved, the probe fluorophore is no longer close to or connected to the quencher moiety, and its fluorescence can no longer be quenched. Other endonucleases such as Apurinic/Apyrimidinic Endonuclease I or Endonuclease IV also recognize the same substrate and cleave such a bound probe in the same way but leave the fragment on the 5′ side of the AP site with a new 3′ hydroxyl that could in theory be extended by a DNA polymerase. However, this would be unlikely as the fragment would quickly dissociate back into solution at the reaction temperature. Additionally, locating a ribobase 6-8 nucleotides from the 5′ end will allow the efficient cleavage by RNase H2 but still be too short of a remaining primer for Bst DNA Polymerase to bind and extend therefrom.

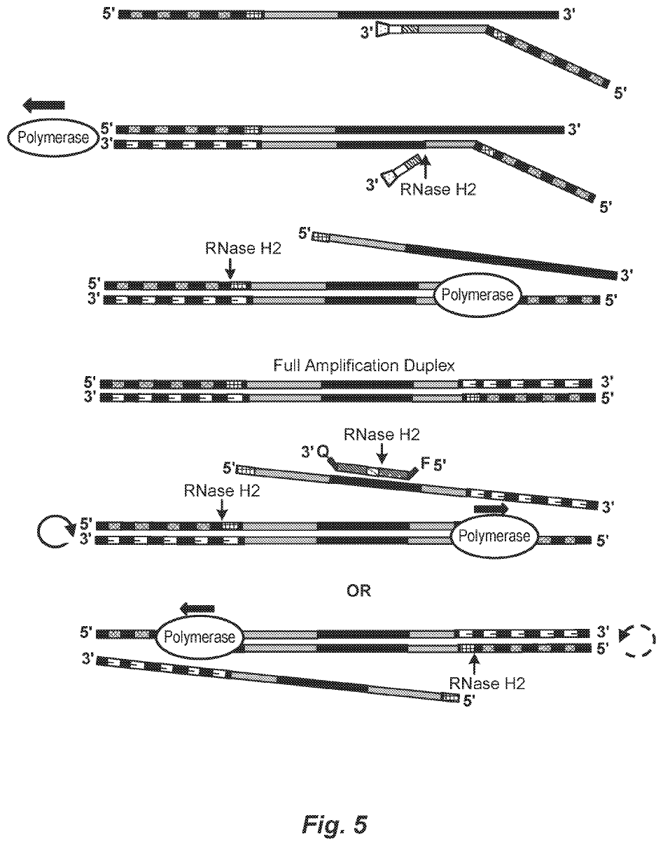

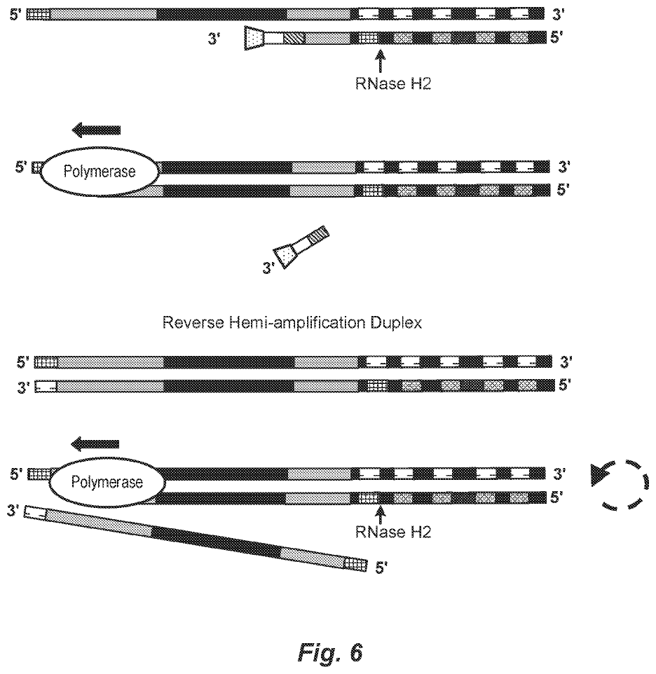

The primers shown in FIG. 1 and FIG. 2 include the forward and reverse primers as described above, which are blocked on the 3′ end until activated. For RNA targets, they can include an additional cDNA synthesis primer that is unblocked and at exceptionally low concentration for initiating first strand synthesis without interacting or interfering too much with other oligos present because of the low concentration. There is also an optional strand displacement initiation primer which is not necessary for exponential amplification, but its presence can further accelerate the initiation of the amplification reaction from a single-stranded DNA target. Alternate embodiments do away with the need for a strand displacement initiation primer by instead utilizing strand invasion of a new primer with a higher T_{M} owing to the modified bases in the 5′ end of the primer. Any number of hybridization probes would work equally well for detection, including linear probes, Eclipse probes, Pleiades probes, Molecular Beacons, and a variety of modified versions of Molecular Beacons. Many hybridization probes are highly temperature dependent and should be designed with care. However, this task is made easier by the constant reaction temperature. The disadvantages of some hybridization probes such as Molecular Beacons in hybridization kinetics associated with fluctuating temperatures in PCR, are also mitigated by the isothermal conditions. One embodiment shown in FIG. 5 employs a linear probe with the modification of a ribobase in the middle of the probe. In linear probes, the probes fold up and quench based on hydrophobic interactions which are less temperature-dependent than the hairpin structure of Molecular Beacons. These linear probes have the ribobase located relatively close to the 5′ end such that RNase H2 activity is maintained, but the 5′ fragment left is too small for extension by Bst polymerase. Careful design ensures good and specific probe hybridization when intact, and quick dissociation of the cleaved fragments.

As with all amplification reactions a complete set of deoxyadenosine triphosphate, deoxycytidine triphosphate, deoxyguanosine triphosphate, and deoxythymidine triphosphate is included at a concentration that is preferably optimized for best performance of each assay design, typically around 200 μM of each. Additionally, the deoxynucleoside triphosphates that are the complementary bases to the non-natural ribobases in the forward and reverse primers allow for their incorporation into the opposing strand complementary to the primer sequence. By incorporating these as deoxynucleoside triphosphates rather than ribonucleoside triphosphates, the potential side reaction of cleaving the very 3′ end by the RNase H2 activity is prevented, and the subsequent incorporation of new non-natural ribobases on the primer strand is templated. In one embodiment presented here, the deoxynucleoside triphosphates would be the 3-β-D-ribofuranosyl-(2,6-diaminopyrimidine) triphosphate and deoxyisocytidine triphosphate. In some embodiments the deoxynucleoside triphosphates could include non-natural nucleotide bases of the group consisting of xanthosine, isoguanosine, deoxyxanthosine, deoxyisoguanosine, isocytosine, deoxyisocytosine, 6-amino-5-nitro-3-(1′-beta-D-2′-ribofuranosyl)-2(1H)-pyridone, 6-amino-5-nitro-3-(1′-beta-D-2′-deoxyribofuranosyl)-2(1H)-pyridone, 2-amino-8-(1′-beta-D-2′-ribofuranosyl)-imidazo[1,2-a]-1,3,5-triazin-4(8H)-one, 2-amino-8-(1′-beta-D-2′-deoxyribofuranosyl)-imidazo[1,2-a]-1,3,5-triazin-4(8H)-one. A convention is assumed here where the “forward” primer is the one that can be used to generate excess target strands for probe detection by asymmetric amplification, although this is completely arbitrary and unrelated to the sense or antisense of the RNA or ssDNA being initially targeted for amplification. Following this convention, the ribonucleoside triphosphate complementary to the non-natural base in the forward primer is included at an optimized concentration with no deoxynucleoside triphosphates of the same base, xanthosine in a first embodiment, present at all. This ensures that all subsequent copies of this strand will incorporate a xanthosine ribobase at the appropriate location and continue to be cleaved by RNase H2 activity, thus providing additional linear amplification of this specific strand for detection. In contrast, as an example, the non-natural base present in the reverse primer, isoguanosine, will have both isoguanosine triphosphate and deoxyisoguanosine triphosphate present, each in optimized concentrations. By varying the concentration of each, and in particular the ratio of the two, one can modulate the probability of incorporating a ribobase versus a standard DNA base at the position opposite the only location of deoxyisocytidine in the full amplicon. While the DNA polymerase greatly prefers to incorporate a deoxynucleoside triphosphate opposite a DNA base, this can be overcome by a number of methods, including a strong bias in the ratio of the availability of ribonucleoside triphosphates to deoxynucleoside triphosphates, the addition of reagent additives such as divalent manganese ions, the choice of specific DNA polymerase, or mutations to the polymerase to reduce its fidelity to DNA versus RNA nucleoside triphosphates. A preferred embodiment presented here is for a strong ratio biasing towards ribonucleoside triphosphates and with the potential addition of a small amount of manganese chloride in addition to the magnesium chloride, with the concentrations to be empirically determined for each assay. The objective is to have the reverse strand continue to support exponential amplification at the fastest speed as possible, while still generating excess of the forward strand by slightly reducing the supplemental linear amplification of the reverse strand. This excess forward strand leads to the rapid detection by the hybridization probe with cycling probe technology. Without this asymmetry, all forward strands would tend to equally pair with reverse strands, and nothing would be available for probe hybridization, even despite millions to even trillions of copies made by the reaction.

A second embodiment is to include only ribonucleoside triphosphates with the non-natural base that is incorporated into the reverse primer, isoguanosine, for example. There would be no deoxyisoguanosine triphosphate present. Rather, the reverse primer would be composed of a mixture of two nearly identical oligonucleotides (“oligos”), one would include an isoguanosine ribobase and the other would include a deoxyisoguanosine DNA base. This could of course be carried out with other non-natural bases instead. Then, during exponential amplification, the reverse strands that were initiated with a reverse primer containing the ribobase would continue to generate reverse strands in linear amplification by RNase H2 cleavage of that ribobase, while reverse strands that were initiated with a reverse primer containing the DNA non-natural base, e.g. deoxyisoguanosine, would only support the completion of the Reverse Hemi-amplification Duplex without any additional linear or exponential amplification from RNase H2 cleavage of the non-natural ribobase. By adjusting the ratio of these two variants of the reverse primer, one could bring about the asymmetric amplification to support good probe hybridization without having to significantly alter the primer concentration itself. This embodiment would work well with complex multiplexed reactions, where the asymmetry could be adjusted individually by not only adjusting primer concentrations, but also adjusting the ratio of the RNA to the DNA versions of the non-natural base in the loop region of the reverse primer for each amplicon in the multiplex set.

A simpler alternative embodiment would be to use only two non-natural bases instead of four. In this embodiment, one ribobase, for example isoguanosine, would be incorporated into the universal adapter of both the forward and reverse primers, while the corresponding deoxynucleoside triphosphate, in this case deoxyisoguanosine triphosphate would be included in the reagents without any corresponding ribonucleoside triphosphate. The complementary non-natural ribonucleoside triphosphate that is the same base as the ribobase included in the universal adapter of both primers would only be provided as a ribonucleoside triphosphate and not as a deoxynucleoside triphosphate. This arrangement would provide for exponential amplification, but asymmetric amplification would only be achieved through either adjusting the primer concentrations or providing a mix of reverse primers wherein some of the reverse primers contain an isoguanosine ribobase and some of the reverse primers contain deoxyisoguanosine at the same location in the primer universal adapter. In this way, not every primer-initiated strand elongation results in a continuation of linear or exponential amplification. Rather, only those reverse strands that have a ribobase in the reverse primer adapter would result in a continuation of linear or exponential amplification. This embodiment would also be well-suited to use in multiplex reactions, as it would allow the direct adjustment of asymmetry independently for each amplicon in the multiplex set.

The buffer components used in amplification reactions are well known to those in the art. The enzyme and oligo interactions presented here can occur under a range of buffer conditions, and depending on enzyme choice and proper oligo design, the reaction can proceed under a number of different temperatures as well. The illustrative embodiment described here, particularly with a Bst DNA polymerase, employs RNase H2, and RTx reverse transcriptase, if necessary, for RNA targets, along with appropriately designed primers and probes, runs ideally at 65° C. The buffer compositions include a pH buffer that has a neutral pH at the reaction temperature, a low amount of ammonium chloride to help reduce non-specific weak hydrogen bonding, a low amount of potassium chloride to stabilize enzymes while not overly stabilizing DNA helices, surfactant for enzyme activity, particularly RNase H2, divalent magnesium, a cofactor for nearly all DNA polymerases and reverse transcriptases, and finally manganese chloride, as necessary, to induce the DNA polymerase to incorporate non-natural ribonucleoside triphosphates efficiently in addition to the incorporation of non-natural deoxynucleoside triphosphates and natural dNTPs.

While the illustrative embodiment has a nominal reaction temperature of 65° C., it should be emphasized that the amplification reaction functions under a broad range of thermal conditions. What is important to the performance is remaining within the active temperature range of all the enzymes present in the reaction. Of course, whichever temperature condition is selected, the T_{M} of the different primers and probes may need to be adjusted accordingly. Moreover, while a single isothermal reaction condition is presented here as the illustrative embodiment, there is nothing to prevent changing the temperature during the reaction, provided the temperature stays within this active range. Like the benefits of Touchdown PCR, one could slowly ramp the temperature down over the course of the reaction to increase specificity of the early binding hybridization events that have a disproportionately large impact on overall assay performance. However, increased specificity can also be achieved by paying more attention to the design and optimization of the primer and probe compositions during the assay design. Likewise, fluctuating or cycling the temperature range within the limits of the active range of the enzymes would still produce amplification and detection by this method, but are not necessary to accomplish this.

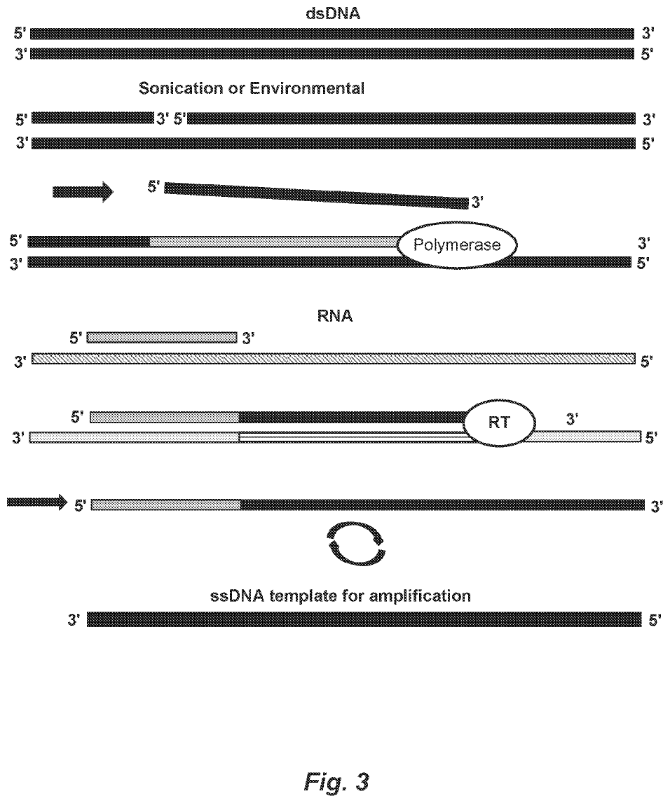

Depending on the source of the RNA or DNA to be detected, the initial formation of single-stranded target DNA for amplification can be made accessible in several ways. For double-stranded DNA, such as from bacteria or humans, the dsDNA can of course be separated by heat denaturation prior to the addition of heat-labile enzymes. A more convenient approach takes advantage of the strand displacement properties of a strand displacing DNA polymerase present in the reaction, whereby any single-stranded nicks or gaps on the 5′ side of the target sequence will naturally be extended without the need for any primer or nuclease. In the process of extension from a nick or gap, the displaced DNA strand will be available for single-stranded DNA target amplification. The presence of nicks or gaps is common in many extracted DNA samples, particularly those that have been exposed to harsh conditions, but for improved speed and performance, additional nicks can be efficiently introduced randomly throughout the genome by exposure to ultrasonication as shown in FIG. 3. Other forms of introducing single-stranded DNA gaps or nicks, either physically, chemically, or enzymatically, would also work.

For RNA targets, such as RNA viruses, human mRNA, bacterial rRNA, or others, a reverse transcriptase can be included to improve initial cDNA synthesis. While cDNA synthesis can occur with the complementary primer with a low rate of RNA cleavage in an RNA: RNA hybrid from RNase H2, this can also be accomplished by including an unblocked cDNA synthesis primer complementary to the region 3′ of the target sequence region. Provided that the reverse transcriptase has RNase H activity, the RNA strand will be hydrolyzed as the cDNA strand is synthesized, leaving a single stranded DNA template for amplification as shown in FIG. 3.

This single-stranded DNA template then can initiate the target-specific amplification process by first incorporating one and then the second primer. The first primer, which is complementary to the single stranded DNA that is available, binds via the target-specific (gene-specific) sequence on the 3′ end on the primer. This complex is recognized by RNase H2, which cleaves the ribobase located in the target-specific (gene-specific) region, thus activating the primer for immediate extension by the DNA polymerase. When the ribobase is cleaved, the blocked 3′ fragment of the primer is released into solution and the nascent DNA strand has newly synthesized normal DNA base pairs.

In some embodiments, the blocked 3′ fragment of the primer can contain a modified high-Tm modified base section.

In some embodiments, another primer can then come in and displace the 5′ end of the nascent strand by strand invasion because of the significant difference in T_{M} of the new primer with modified high T_{M} bases and the natural DNA base pairs. Activation and extension from this second primer then displaces the previously synthesized strand with the 5′ end of the first primer attached, which can be either the forward or reverse primer depending on the complementarity of the original single-stranded DNA template for amplification.

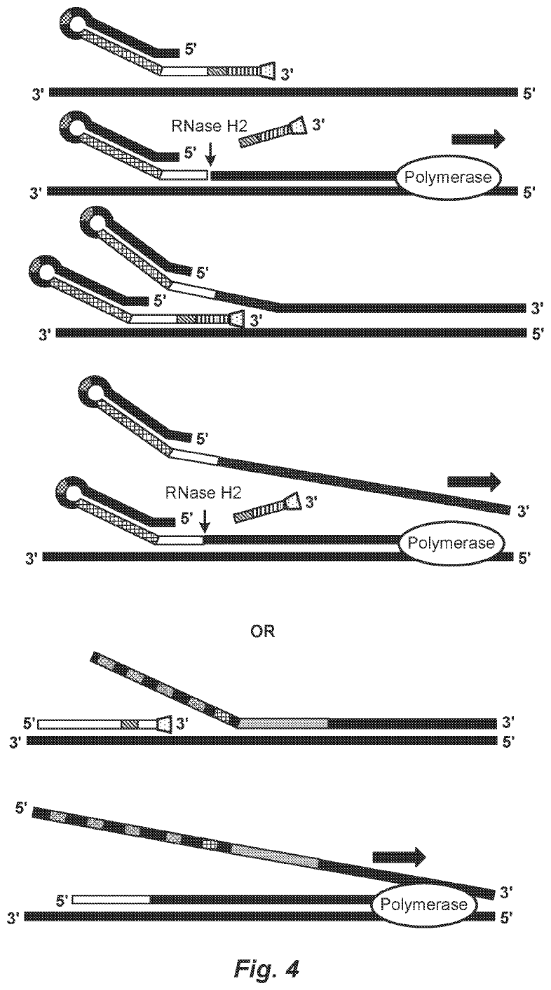

In some embodiments this process includes the addition of another strand displacement initiation primer to the reaction mixture. This additional primer would be blocked as well, at a lower concentration than the forward primer, and would contain a target-specific (gene-specific) ribobase. The sequence would be complementary to the sequence that is 3′ of the target sequence region, as shown in FIG. 4. Because of its lower concentration than the forward primer, the forward primer would be kinetically favored to bind and extend first before the strand displacement initiation primer. (The difference between the concentrations of the forward and initiation primers can be determined empirically for particular applications.) When the strand displacement initiation primer did then bind, activate, and extend, it would displace the initial nascent DNA target strand with the 5′ end of the first primer attached. This alternate embodiment can provide a benefit in terms of faster initiation than the approach mediated by strand invasion.

This newly created single-stranded DNA which is capped at its 5′ end with one of the two primer sequences would then be available to bind the other primer. In this example shown in FIG. 5, when the reverse primer is bound, activated by RNase H2, and extended, the strand displacing polymerase will create the forward primer's double-stranded DNA complement including the universal adapter sequence with non-natural bases. The non-natural ribobase held in the adapter region would then become part of the double stranded amplification product, with its complementary non-natural DNA base in the opposing strand. This ribobase, while it is protected from RNase H2 cleavage when single-stranded, would then be recognized and cleaved by RNase H2. Extension from this cleavage site would displace the existing DNA strand which only had one primer sequence attached and create a new Full Amplification Duplex structure consisting of a double-stranded DNA amplicon of the target sequence flanked by the forward and reverse primers including the very 5′ ends of the universal adapter in each primer. Because of the non-natural base in the strand complementary to the primer, the corresponding non-natural ribobase in the adapter of the primer, either isoguanosine or xanthosine, for example, no deoxynucleoside triphosphates for this position are provided in the reaction. Instead, the corresponding ribonucleoside triphosphates are provided for incorporation. While the Bst DNA polymerase has a strong preference for the incorporation of dNTPs, it does have limited ability to incorporate rNTPs although much less efficiently. The ability to incorporate a non-natural rNTP can be further enhanced by mutation of the enzyme or addition of reagents such as manganese ions. Thus, as a new strand of DNA is synthesized from the RNase H2 cleavage and strand extension from the new 3′ hydroxyl that is formed, a new single ribobase is also incorporated into the new strand at the position that the non-natural ribobase was located in the primer. This enables the repetitive cleavage and extension of this specific strand of the target DNA in a linear amplification method.

The reverse strand follows the same process and provides separate linear amplification of the complementary strand. The rate of linear amplification of each of these strands from the Full Amplification Duplex can be modulated in several ways. In some embodiments, different non-natural bases can be incorporated into the forward and reverse primers, with the supplement of deoxynucleoside triphosphates in addition to ribonucleoside triphosphates, which will serve to reduce the amount of linear amplification from this strand depending on whether a non-natural DNA base is incorporated or a non-natural RNA base is incorporated. Only an incorporated RNA base will lead to further linear amplification. Despite a strong bias by the polymerase for dNTP incorporation, a strong bias in ribonucleoside triphosphate to deoxynucleoside triphosphate concentration for the corresponding non-natural base can compensate and adjust for this bias. Second, the concentration of the forward and reverse primers can be adjusted independently, and biases in the concentration of forward and reverse primers can then drive asymmetric amplification of the two strands. Third, either or both primers can actually be composed of a mixture of oligonucleotide primers wherein the same sequence and structure is used for each, and only the presence of a non-natural ribobase or its DNA equivalent is incorporated into the adapter region of the primer. In an illustrative embodiment, this would be where the forward primer, which is arbitrarily designated as the primer that initiates the strand that is complementary to the detection probe, is provided only with the ribobase variant. The reverse primer could then be supplied as mostly consisting of the ribobase variant to support linear and exponential amplification, and then supplemented with an empirically derived concentration of the non-natural DNA variant to bring about asymmetric amplification of the forward strand. The latter approaches to adjustments solely in the concentration or composition of the reverse primer are well suited to highly multiplexed reactions, where each amplicon in the full set can be individually adjusted for optimal asymmetric amplification and detection. Changes in either the ribonucleoside triphosphate or primer sequence have the advantage of kinetically favorable primer concentrations for the reverse primer binding to its complementary sequence, which is a known disadvantage of traditional asymmetric amplification techniques that rely solely on greatly reducing one primer concentration relative to the other.

The asymmetric amplification of one strand, in this case the forward strand, is essential for rapid and efficient detection of the target sequence by a hybridization probe. By driving the excess amplification of one strand over the other, there will always be some unhybridized single-stranded amplicon in solution and available for hybridization and detection by the probe. The probe can bind to the sequence at other times as well, such as immediately after the forward strand is displaced by the linear amplification and before it has a chance to hybridize with a free complementary strand. Promoting sufficient amplification asymmetry will ensure efficient probe detection. Furthermore, the isothermal amplification conditions are ideally suited to the incorporation of a cycling probe technology as described above to further accelerate detection.

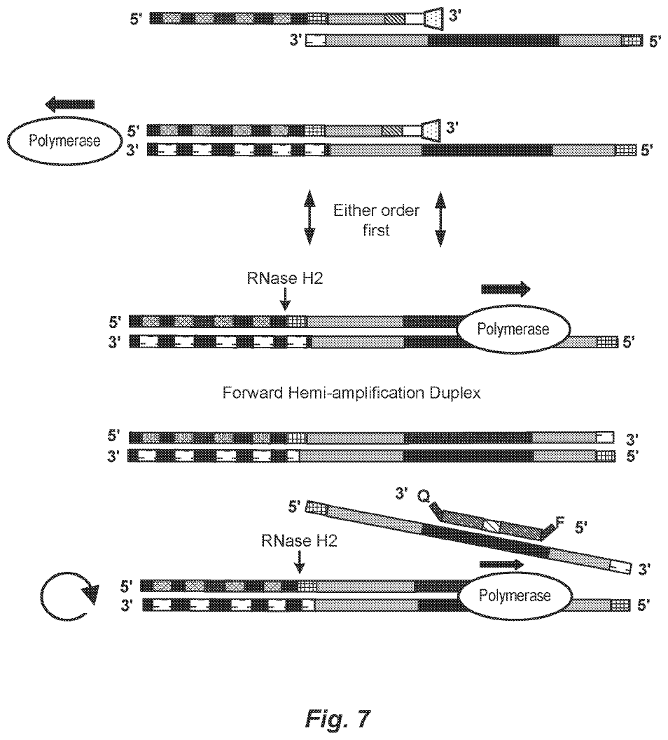

When each strand is displaced, it becomes available to be bound by the complementary primer in solution. For instance, the reverse strand displaced from the cleavage of the ribobase in the reverse primer would then bind to the complementary forward primer when it was available and free in solution, as shown in FIG. 7. In this case, the reverse strand would contain the sequence of the reverse primer starting with the non-natural ribobase and proceeding in the 3′ direction all the way through the target sequence, and then the natural bases that are complementary to the full forward primer sequence including the linearized adapter sequence. At the same time and independently, the adapter sequence in the forward primer would also have its exact complement in the reverse strand, along with the complement of the non-natural ribobase and the unpaired bases at the very 5′ end of the primer. The full double-stranded structure known as the Forward Hemi-amplification Duplex is so named because the full sequence of the forward primer is included, along with its complement, but only the fragment of the reverse primer that includes the non-natural ribobase and the sequence 3′ of that, along with the full complementary strand. Thus, only the forward sequence has the full original primer sequence including the 5′ universal adapter to allow RNase H2 cleavage of the non-natural ribobase. As this site is cleaved, the DNA polymerase will extend from the new 3′ hydroxyl created—starting with another non-natural ribobase—and then proceed down the full length of the amplicon. The displaced strand would be immediately available for probe hybridization but could also participate in several other reactions as described below. As the ribobase is immediately replaced upon excision, this process would repeat indefinitely in a linear amplification of this strand.

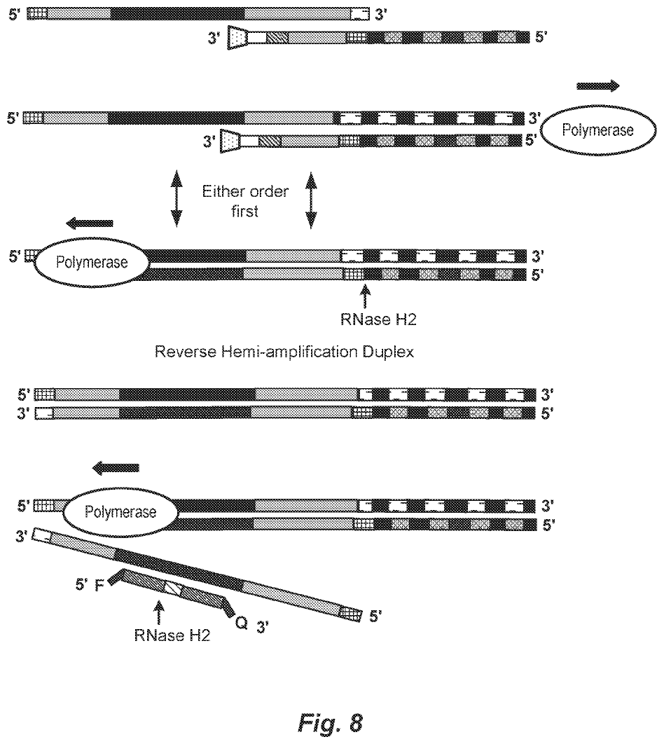

When the forward strand is displaced from the Full Amplification Duplex, it becomes available to be bound by the reverse primer. FIG. 8 describes the same process as in FIG. 7, only for the opposing strand with the reverse primer. This leads to the formation of the Reverse Hemi-amplification Duplex. The repeating cleavage of the non-natural ribobase leads to the linear amplification of the reverse strand target sequence. Simply by naming convention, this strand does not participate in probe binding and detection. The cleavage of the non-natural ribobase in this Reverse Hemi-amplification Duplex can be modulated with any of the previously described methods for rNTP/dNTP adjustments, skewed primer concentration ratios, or mixtures of reverse primer with or without non-natural ribobases.

The target DNA strands produced by RNase H2 cleavage and DNA polymerase extension of the Forward and Reverse Hemi-amplification Duplexes yield a single-stranded product that is bound on both ends by the non-natural bases—with the non-natural ribobase at the 5′ end and a non-natural DNA base at the 3′ end—with the full intervening sequence. FIG. 7 shows the forward primer binding onto the complementary sequence of the reverse strand. Independently, two different events will then occur in either order: 1) the target-specific (gene-specific) ribobase will be recognized and cleaved by RNase H2, followed by strand extension from the DNA polymerase; and 2) the 5′ overhang of the universal adapter sequence will provide a template for extension from the free 3′ hydroxyl of the target DNA strand. When both steps are completed, it will make a complete Forward Hemi-amplification Duplex as shown in FIG. 7. As described earlier, this structure would provide for linear amplification, exponential amplification, and detection. The exact same process is shown in FIG. 8 for the forward strand with the reverse primer leading to the formation of the Reverse Hemi-amplification Duplex, which can also participate in linear and exponential amplification as well as be modulated for asymmetric amplification.

Both of the single-stranded DNA products from the linear amplification of the Forward and Reverse Hemi-amplification Duplexes will most likely bind to the opposing primer sequence because of the great difference in concentration between the primers and the initial concentration of the amplification products. In this way, they support the exponential amplification of the target sequence. However, it is entirely possible, particularly later in the reaction as it progresses, that two complementary DNA strands will find each other and bind together to form a double-stranded DNA molecule with only the target sequence that lies between the non-natural ribobases on both ends. The formation of this structure is limited by the presence of the lower concentration of the two molecules, relative to both primer concentrations and the more abundant strand. Thus, the presence of this structure is limited by the asymmetry of the amplification reaction, leaving the forward strand available for detection. When these complementary strands do hybridize, the lack of any DNA to the 5′ side of either the forward or reverse ribobase means that there is no substrate for RNase H2 to act upon, and consequently no free 3′ hydroxyl from which to extend for the DNA polymerase.

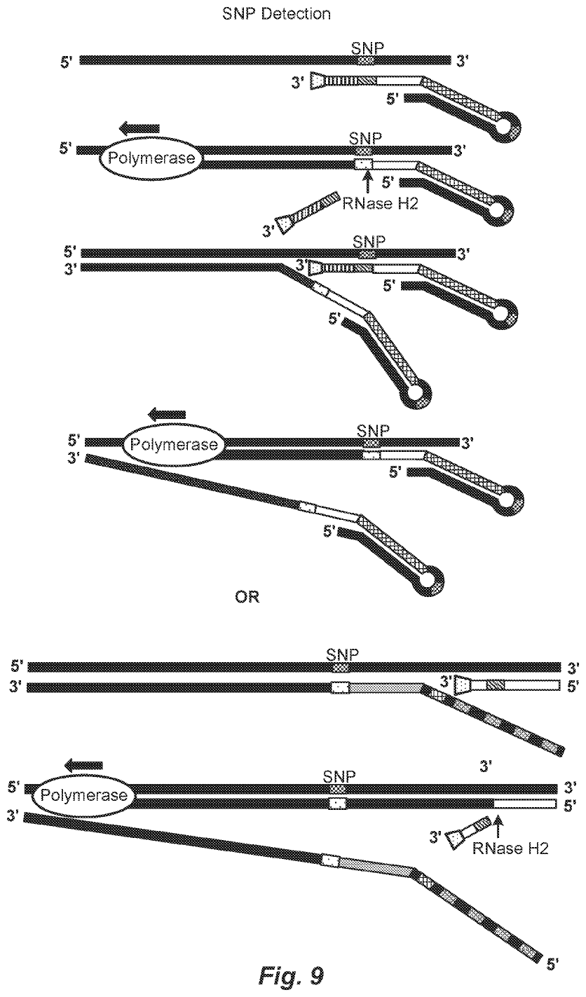

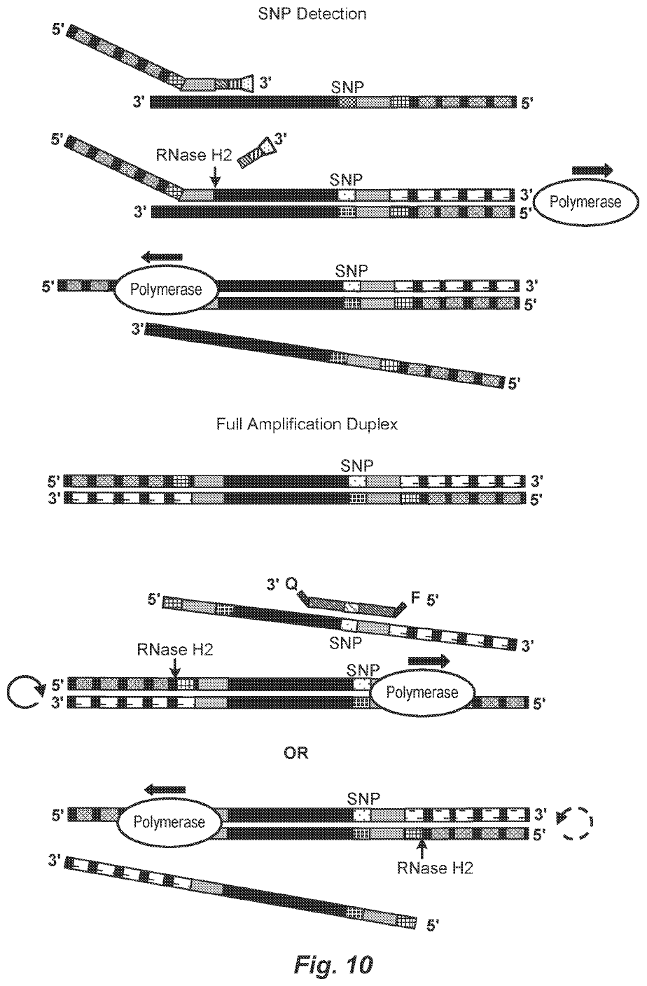

In some embodiments, this amplification and detection chemistry system can be used to detect and differentiate Single Nucleotide Polymorphisms (SNPs) in a rapid, specific, and sensitive manner. While many SNPs are highly relevant to several different human diseases and risk factors for disease progression such as many cancers, they are often challenging and cumbersome to reliably detect and differentiate. One main reason for this is that it is not merely a matter of presence or absence of an entire target sequence, but rather a single base difference between two alternate alleles. The current system can utilize the extremely high specificity of the RNase H2 enzyme activity for a ribobase that is correctly paired with its complementary base as a way to discriminate between SNPs. Following the convention of arbitrarily labeling the strand with excess amplification as the forward strand, the reverse primer is designed so that a SNP of interest is located exactly opposite the target-specific (gene-specific) ribobase of the reverse primer when this primer is hybridized. Amplification will only initiate an amplification reaction as shown in FIG. 9 and will only proceed in generating a Full Amplification Duplex, and subsequent linear and exponential amplification as shown in FIG. 10, if the reverse primer is an exact match to the SNP following the same process described above. Another embodiment of this approach to SNP detection and differentiation would facilitate high performance in rare allele detection. Often, when a sample contains a mixture of allelic variants at vastly different concentrations, such as the presence of a tumor-associated mutation, it can be difficult to detect the rare allele amongst the abundant background amplifying and consuming reaction materials. Beyond just identifying and discriminating between alternate variants of an allele, a rare allele can be specifically targeted by not including the predominant allele-specific primer. Thus, amplification only occurs if the rare allele is present, even if the concentration is extremely low.

The probe to detect the presence of the target SNP must be the same sense as one of the primers, in this case the same sense as the reverse primer, in order to prevent a false positive detection by binding to a primer rather than an amplicon. Only after successful amplification would sufficient amplicon that is complementary to the reverse primer and probe, be present to facilitate detection by the probe. Moreover, the primers designed for SNP discrimination could have different universal adapter sequences, such that each different SNP probe would target the junction between the target-specific (gene-specific) region, including the SNP of interest, and the SNP-specific adapter sequence. This would provide much more differentiation and resolution of the different SNPs that were targeted, beyond just the single nucleotide.

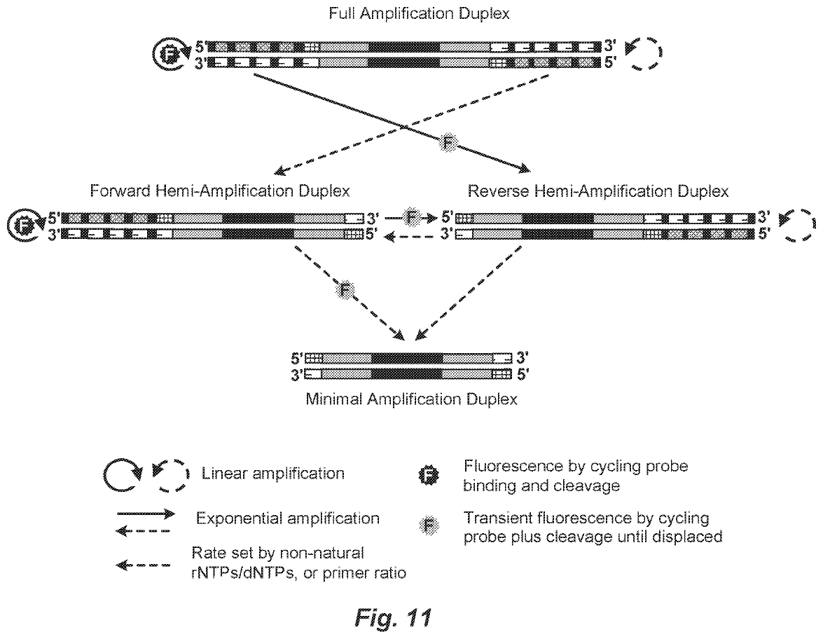

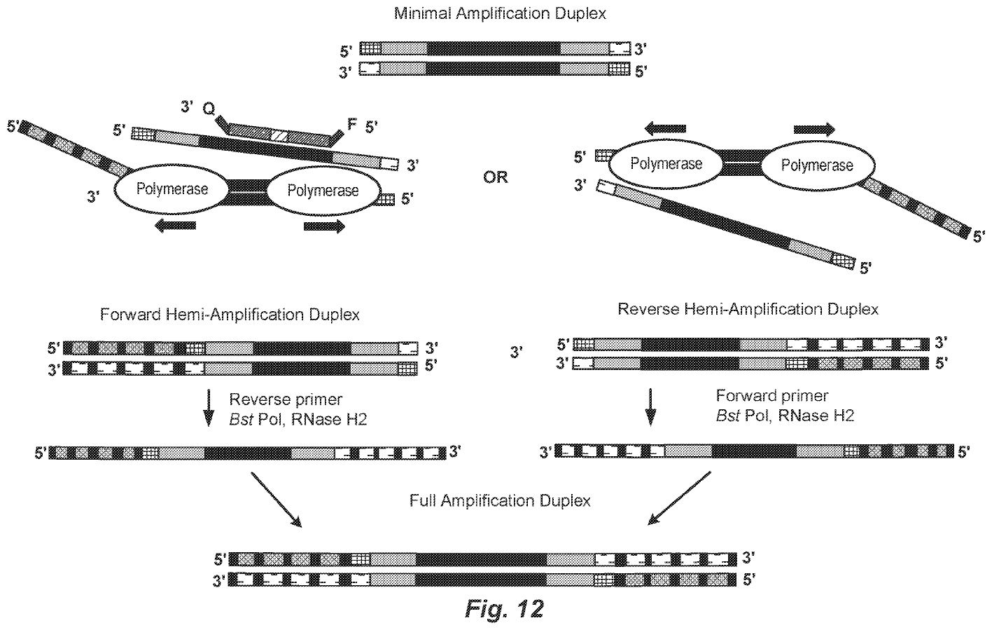

FIG. 11 and FIG. 12 provide a schematic summary of the various amplification products and pathways that can occur within this whole isothermal system. Much like the old proverb “there are many paths to the top of the mountain, but the view is always the same”, this comprehensive system for amplification and detection has multiple paths for linear amplification shown with the circular arrows and exponential amplification with the horizontal arrows. Each strand's amplification can be controlled independently for both the forward (solid arrows) and reverse (dashed arrows) strands though multiple paths including different non-natural NTPs and dNTPs, primer mixtures of non-natural ribobases and deoxynucleoside bases, or simply differential primer concentrations. Inevitably, many reaction chemistries lose efficiency as the reaction progresses when the products become more abundant and the probability of encountering a complementary product before primer binding and extension can happen. In this system, that is restricted to less than the abundance of the reverse strand, leaving the excess of the forward strand available for continued detection. With the incorporation of cycling probe technology in the probe design, the excess of the forward strand will continue to generate more fluorescence signal for detection as the reaction proceeds, even after the end of exponential or linear amplification, or the consumption of other reaction components such as primers and dNTPS. By design, all of these amplification and detection pathways, and their constituent chemistries, will ensure the entire system-level performance to deliver the purpose-built performance characteristics of minimal sample preparation, rapid time to result, high performance on sensitivity and specificity, and full compatibility with high-level multiplexing.

### Samples

Nucleic acid-containing samples can be obtained from biological sources and prepared using conventional methods known in the art. In particular, nucleic acids useful in the methods described herein can be obtained from any source, including unicellular organisms and higher organisms such as plants or non-human animals, e.g., canines, felines, equines, primates, livestock (sheep, cattle, and pigs) and other non-human mammals, as well as humans. In some embodiments, samples may be obtained from an individual suspected of being, or known to be, infected with a pathogen, an individual suspected of having, or known to have, a disease, such as cancer, or a pregnant individual.

Nucleic acids can be obtained from cells, bodily fluids (e.g., blood, a blood fraction, urine, etc.), or tissue samples by any of a variety of standard techniques. In some embodiments, the method employs samples of plasma, serum, spinal fluid, lymph fluid, peritoneal fluid, pleural fluid, oral fluid, and external sections of the skin; samples from the respiratory, intestinal genital, or urinary tracts; samples of tears, saliva, blood cells, stem cells, or tumors. Samples can be obtained from live or dead organisms or from in vitro cultures. Illustrative samples can include single cells, paraffin-embedded tissue samples, and needle biopsies. In some embodiments, the nucleic acids analyzed are obtained from a single cell.

Nucleic acids of interest can be isolated using methods well known in the art. The sample nucleic acids need not be in pure form but are typically sufficiently pure to allow the steps of the methods described herein to be performed.

### Target Nucleic Acids

Any target nucleic acid that can detected by nucleic acid amplification can be detected using the methods described herein. In typical embodiments, at least some nucleotide sequence information will be known for the target nucleic acids. For example, if the amplification reaction employed is PCR, sufficient sequence information is generally available for each end of a given target nucleic acid to permit design of suitable amplification primers.

The targets can include, for example, nucleic acids associated with pathogens, such as viruses, bacteria, protozoa, parasites or fungi; RNAs, e.g., those for which over- or under-expression is indicative of disease, those that are expressed in a tissue- or developmental-specific manner; or those that are induced by particular stimuli; genomic DNA, which can be analyzed for specific polymorphisms (such as SNPs), alleles, or haplotypes, e.g., in genotyping. Of particular interest are genomic DNAs that are altered (e.g., amplified, deleted, and/or mutated) in genetic diseases or other pathologies; sequences that are associated with desirable or undesirable traits; and/or sequences that uniquely identify an individual (e.g., in forensic or paternity determinations).

### Primer Design

Primers suitable for nucleic acid amplification are sufficiently long to prime the synthesis of extension products in the presence of a suitable nucleic acid polymerase. The exact length and composition of the primer will depend on many factors, including, for example, temperature of the annealing reaction, source and composition of the primer, and where a probe is employed, proximity of the probe annealing site to the primer annealing site and ratio of primer:probe concentration. For example, depending on the complexity of the target nucleic acid sequence, an oligonucleotide primer typically contains in the range of about 10 to about 60 nucleotides, although it may contain more or fewer nucleotides. The primers should be sufficiently complementary to selectively anneal to their respective strands and form stable duplexes.

In general, one skilled in the art knows how to design suitable primers capable of amplifying a target nucleic acid of interest. For example, PCR primers can be designed by using any commercially available software or open-source software, such as Primer3 (see, e.g., Rozen and Skaletsky (2000) Meth. Mol. Biol., 132:365-386; www.broad.mit.edu/node/1060, and the like) or by accessing the Roche UPL website. The amplicon sequences are input into the Primer3 program with the UPL probe sequences in brackets to ensure that the Primer3 program will design primers on either side of the bracketed probe sequence.

Primers may be prepared by any suitable method, including, for example, direct chemical synthesis by methods such as the phosphotriester method of Narang et al. (1979) Meth. Enzymol. 68:90-99; the phosphodiester method of Brown et al. (1979) Meth. Enzymol. 68:109-151; the diethylphosphoramidite method of Beaucage et al. (1981) Tetra. Lett., 22:1859-1862; the solid support method of U.S. Pat. No. 4,458,066 and the like, or can be provided from a commercial source. Primers may be purified by using a Sephadex column (Amersham Biosciences, Inc., Piscataway, NJ) or other methods known to those skilled in the art. Primer purification may improve the sensitivity of the methods described herein.

### Polymerase

The disclosed methods make the use of a polymerase for amplification. In some embodiments, the polymerase is a DNA polymerase that lacks a 5′ to 3′ exonuclease activity. The polymerase is used under conditions such that the strand extending from a first primer can be displaced by polymerization of the forming strand extending from a second primer that is “outer” with respect to the first primer. Conveniently, the polymerase is capable of displacing the strand complementary to the template strand, a property termed “strand displacement.” Strand displacement results in synthesis of multiple copies of the target sequence per template molecule. In some embodiments, the DNA polymerase for use in the disclosed methods is highly processive. Exemplary DNA polymerases include variants of Taq DNA polymerase that lack 5′ to 3′ exonuclease activity, e.g., the Stoffel fragment of Taq DNA polymerase (ABI), SD polymerase (Bioron), mutant Taq lacking 5′ to 3′ exonuclease activity described in U.S. Pat. No. 5,474,920, Bca polymerase (Takara), Pfx50 polymerase (Invitrogen), Tfu DNA polymerase (Qbiogene). If thermocycling is to be carried out (as in PCR), the DNA polymerase is preferably a thermostable DNA polymerase. Table 2 below lists polymerases available from New England Biolabs that have no 5′ to 3′ exonuclease activity, but that have strand displacement activity accompanied by thermal stability.

TABLE A
Thermostable Stand-Displacing Polymerases Lacking
5′ to 3′ Exonuclease Activity

| | 5′->3′ | Strand | Thermal |
|---|---|---|---|
| Polymerase | Exonuclease | Displacement | Stability |
| Bst DNA Polymerase, | − | ++++ | + |
| Large Fragment | | | |
| Bsu DNA Polymerase, | − | ++ | − |
| Large Fragment | | | |
| DEEP VENT_{R} ™ | − | ++ | ++++ |
| DNA Polymerase | | | |
| DEEP VENT_{R} ™ (exo-) | − | +++ | ++++ |
| DNA Polymerase | | | |
| Klenow Fragment (3′→5′ exo-) | − | +++ | − |
| DNA Polymerase I, | − | ++ | − |
| Large (Klenow) Fragment | | | |
| M-MuLV Reverse | − | +++ | − |
| Transcriptase | | | |
| phi29 DNA Polymerase | − | +++++ | − |
| THERMINATOR ™ DNA | − | + | ++++ |
| Polymerase | | | |
| VENT_{R} ® DNA | − | ++^{e} | +++ |
| Polymerase | | | |
| VENT_{R} ® (exo-) | − | +++^{e} | +++ |
| DNA Polymerase | | | |

In some embodiments, the DNA polymerase comprises a fusion between Taq polymerase and a portion of a topoisomerase, e.g., TOPOTAQ™ (Fidelity Systems, Inc.).

Strand displacement can also be facilitated through the use of a strand displacement factor, such as a helicase. Any DNA polymerase that can perform strand displacement in the presence of a strand displacement factor is suitable for use in the disclosed method, even if the DNA polymerase does not perform strand displacement in the absence of such a factor. Strand displacement factors useful in the methods described herein include BMRF1 polymerase accessory subunit (Tsurumi et al., J. Virology 67(12): 7648-7653(1993)), adenovirus DNA-binding protein (Zijderveld and van der Vliet, J. Virology 68(2): 1158-1164(1994)), herpes simplex viral protein ICP8 (Boehmer and Lehman, J. Virology 67(2): 711-715(1993); Skaliter and Lehman, Proc. Natl. Acad. Sci. USA 91(22): 10665-10669(1994)), single-stranded DNA binding proteins (SSB; Rigler and Romano, J. Biol. Chem. 270:8910-8919(1995)), and calf thymus helicase (Siegel et al., J. Biol. Chem. 267:13629-13635 (1992)). Helicase and SSB are available in thermostable forms and therefore suitable for use in PCR.

### Cycling Probes

In some embodiments, a cycling probe can be used for detection and, optionally, quantification of target nucleic acids in the methods described herein. Cycling probes have been used for years as a way of amplifying signal in amplification assays. Cycling probes are described in, e.g., PCT Publication No. WO 89/09284, and U.S. Pat. Nos. 5,011,769 and 4,876,187, which are incorporated herein by reference for this description.

U.S. Pat. No. 5,763,181 describes the use of fluorescently labeled cycling probes to detect target nucleic acids. Generally, the disclosed method employs a fluorescently labeled oligonucleotide substrate containing a nucleotide sequence that is recognized by the enzyme that catalyzes the cleavage reaction. The oligonucleotide substrate can be DNA or RNA and can be single- or double-stranded. The oligonucleotide can be labeled with a single fluorescent label or with a fluorescent pair (donor and acceptor) on a single strand of DNA or RNA. The choice of single- or double-label can depend on the efficiency of the enzyme employed in the method of the disclosure. There is no limitation on the length of the oligonucleotide substrate, so long as the fluorescent probe is labeled sufficiently far (e.g., 6-7 nucleotides) away from the enzyme cleavage site. Examples of fluorophores commonly used in this method include fluorescein isothiocyanate, fluorescein amine, eosin, rhodamine, dansyl, and umbelliferone. Other fluorescent labels will be known to the skilled artisan. Some general guidance for designing sensitive fluorescently labeled polynucleotide probes can be found in Heller and Jablonski's U.S. Pat. No. 4,996,143. This patent discusses parameters that can be considered when designing fluorescent probes. The cycling probe cleavage reaction can be catalyzed by such enzymes as DNases, RNases, helicases, exonucleases, restriction endonucleases, or retroviral integrases. Other enzymes that effect nucleic acid cleavage are known to the skilled artisan and can be employed to cleave cycling probes having their cognate cleavage sites.

In some embodiments, one or more modified bases can be included in any of the probes described herein. The considerations discussed above regarding the use of stabilizing and/or modified bases in probes also applies to probes.

In some embodiments, it may be convenient to include labels on one or more of the primers employed in in amplification mixture.

### Exemplary Automation and Systems

In some embodiments, a target nucleic acid is detected using an automated sample handling and/or analysis platform. In some embodiments, commercially available automated analysis platforms are utilized. For example, in some embodiments, the GeneXpert® system (Cepheid, Sunnyvale, CA) is utilized.

The methods described herein are illustrated for use with the GeneXpert system. Exemplary sample preparation and analysis methods are described below. However, the present disclosure is not limited to a particular detection method or analysis platform. One of skill in the art recognizes that any number of platforms and methods may be utilized.

The GeneXpert® utilizes a self-contained, single use cartridge. Sample extraction, amplification, and detection may all be carried out within this self-contained “laboratory in a cartridge” (available from Cepheid—see www.cepheid.com).

Components of the cartridge include, but are not limited to, processing chambers containing reagents, filters, and capture technologies useful to extract, purify, and amplify target nucleic acids. A rotatable valve enables fluid transfer from chamber to chamber and contains nucleic acids lysis and filtration components. An optical window enables real-time optical detection. A reaction tube extending from the body of the cartridge enables very rapid thermal cycling.

In some embodiments, the GeneXpert® system includes a plurality of modules for scalability. Each module includes optical and thermal components for amplification and detection, along with mechanical components for sample preparation and controlling fluidic movements in the cartridge.

After the sample is added to the cartridge, the sample is contacted with lysis buffer and released nucleic acid is bound to a nucleic acid-binding substrate such as a silica or glass substrate. The sample supernatant is then removed, and the nucleic acid is eluted in an elution buffer such as a Tris/EDTA buffer. The eluate may then be processed in the cartridge to detect target genes as described herein. In some embodiments, the eluate is used to reconstitute at least some of the reagents, which are present in the cartridge as lyophilized reagents.

In some embodiments, PCR is used to amplify and detect the presence of one or more target nucleic acids. In some embodiments, the PCR uses Taq polymerase with hot start function, such as AptaTaq (Roche).

In some embodiments, an off-line centrifugation is used to improve assay results from samples with low cellular content. The sample, with or without the buffer added, is centrifuged and the supernatant removed. The pellet is then resuspended in a smaller volume of supernatant, buffer, or other liquid. The resuspended pellet is then added to a GeneXpert® cartridge as previously described.

### Fusion Proteins

Fusion enzymes that incorporate both polymerases and endonucleases can be used for various applications (PCR, isothermal amplification, ligation, etc.). The polymerase and endoribonuclease are not restricted to those described in this disclosure. The replicative polymerase can be selected from family A (T7 polymerase, Taq polymerase, etc.) as well as family B (9° N polymerase, Phi 29 polymerase, Pfu polymerase, Vent polymerase). A variety of nucleases can also be employed for either sequence-specific cleavage of hybrid nucleic acid structures (RNA: DNA or abasic lesions) such as Endo IV, SSIII, SSIV, Type I or Type II RNase H. Reverse transcription enzymes are also envisioned as a potential for fusion options or through the use of polymerases such that nascent RT activity is sufficient for target detection. Examples of RT enzymes are EIAV or M-MLV mutants or HIV-1. The fusion of disparate functions can produce a single enzyme with amplification properties that operate at temperatures ranging from 37° C. to 75° C. Amplification can also occur through temperature cycling or isothermally.

We have designed a set of fusion enzymes for in vitro isothermal amplification of nucleic acids integrating added specificity through the use blocked primers. The recombinant fused enzymes include the bacterial DNA polymerase 1 from Geobacillus stearothermophilus large fragment (Bst LF) that is lacking 3′→5′ proofreading exonuclease activity, 5′→3′ exonuclease activity, and RNase H2 from Pyrococcus abyssi. Participation of an endoribonuclease in the amplification scheme ensures that a modified oligonucleotide does not participate in the reaction until it is hybridized and cleaved to produces a functional 3′-hydroxyl end. This added level of control, in comparison to non-modified oligonucleotides, enhances the specificity of the reaction. This disclosure provides end-to-end enzyme chimeras comprising of a DNA polymerase fragment and an endoribonuclease. The endoribonuclease domain is fused to either the C-terminal or N-terminal end of the polymerase domains. In some embodiments the enzymes are fused with an affinity tag and a cleavage sequence on either the N-terminal or C-terminal end of the fusion construct.

In some embodiments, the fusion protein can be a chimera in which one polypeptide is a DNA- or RNA-dependent polymerase and the other functional subunit is an endoribonuclease. The DNA- or RNA-dependent polymerases can have, but do not require, exonuclease, endonuclease, or transferase functions. The endonuclease domain of the said chimera can be oriented such that fusion is done at either the C-terminal or N-terminal of the polymerase domain. Furthermore, the chimera can contain an additional affinity tag with a cleavage domain. The affinity tag can be placed on any terminal domain of the chimera. For example, it can be placed on the N-terminus of the polymerase or endonuclease or placed on the C-terminus of the polymerase or endonuclease. The chimera has both polymerase and endonuclease activity and does not exist in nature (i.e. it is not naturally occurring).

The present invention relates to bridging polynucleotide sequences from the genome of Pyrococcus abyssi and Bacillus stearothermophilus (Geobacillus stearothermophilus) generating a novel enzyme with disparate functions that is not found in nature. The amino acid sequence encoded by the polynucleotide sequence produces a novel enzyme having endoribonucleolytic and polymerase activity and retains activity of both functions at temperatures up to 65° C.

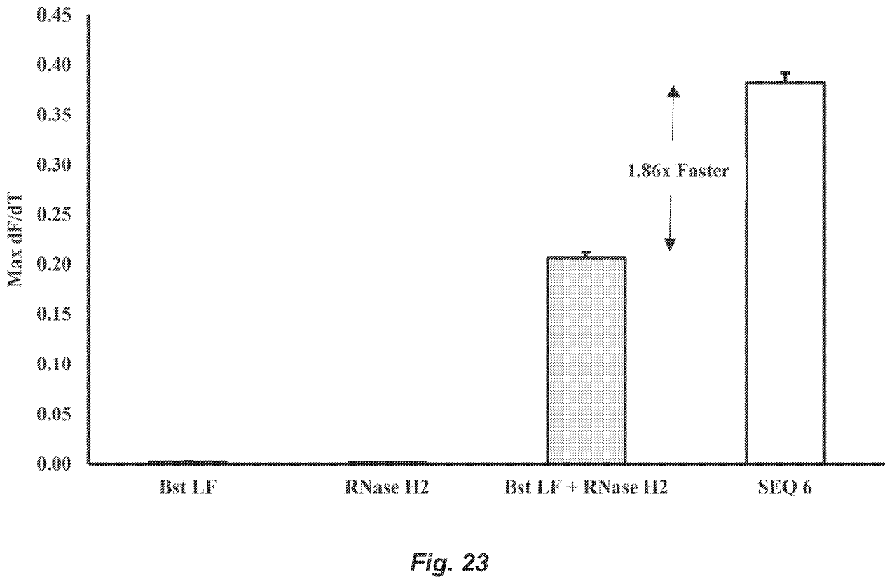

Assembly of the chimeras described herein is accomplished using the Bst DNA polymerase large fragment (Bst LF). The full length Bst DNA polymerase from G. stearothermophilus retains 5′→3′ exonuclease activity but not the 3′→5′ exonuclease activity and has 876 amino acid residues (SEQ ID 1). This embodiment employs a fragment of the full-length DNA polymerase from G. stearothermophilus that does not have 5′→3′ exonuclease activity, lacks 289 amino acid residues from the N-terminal domain, and consists of 587 amino acid residues termed Bst LF (SEQ ID 2; Gene accession U33536.1). The second characteristic desired herein is an endoribonuclease activity which is accomplished by fusing Type II RNase H from Pyrococcus abyssi (SEQ ID 3) with Bst LF. The sequences were obtained from public databases and codon optimized for expression in Escherichia coli. The synthetic genes were inserted into a pET 21b (+) expression vector with 5′ NdeI and 3′ Xhol restriction sites (GENEWIZ) that introduces a polyhistidine sequence into the open reading frame. A TEV protease cleavage site was introduced either upstream or downstream of the histidine sequence as a means for affinity tag removal. Those skilled in the art will appreciate that an affinity tag is not required in order to produce the chimeras and the sequences noted are not intended to limit the scope of the invention to the use of affinity tags. Underlined amino acid sequences throughout this disclosure indicate linker sequences while amino acids in bold indicate affinity tag sequences. In one embodiment, the chimera is oriented such that RNase H2 replaces the 5′→3′ exonuclease domain of Bst DNA polymerase, includes an N- or C-terminal His-TEV cleavage sequence and a flexible linker (SEQ IDs 4, 5). In yet another embodiment, the chimera contains a rigid linker with an N- or C-terminal TEV-His affinity tag (SEQ IDs 6, 7). Further embodiments are fusion enzymes with the endoribonuclease structurally positioned on the C-terminus of the DNA polymerase with an affinity tag located on either the N- or C-terminus of the fusion enzymes that are joined with a flexible linker (SEQ IDs 8, 9). In addition, the chimera with RNase H2 fused to the C-terminus of Bst LF by a rigid linker can have a cleavable affinity tag located on either the N-terminal or C-terminal end (SEQ IDs 10, 11). The two peptide linkers used to fuse Bst LF and RNase H2 consist of either a flexible or helical linker and are listed as SEQ ID 12 and SEQ ID 13 respectively. Those skilled in the art will be able to understand the drawings or schematics presented and are for illustration purposes only and are not intended to limit the scope of the disclosure. The substrates listed as SEQ ID NOs: 14-16 or employed in generating the results of FIG. 23 are not limited to native DNA or RNA bases, any modification to the base, sugar or backbone structures can be used to modulate certain properties of the substrate.

### Kits

Also contemplated is a kit for carrying out the methods described herein. Such kits include one or more reagents useful for practicing any of these methods. A kit generally includes a package with one or more containers holding the reagents, as one or more separate compositions or, optionally, as an admixture where the compatibility of the reagents will allow. The kit can also include other material(s) that may be desirable from a user standpoint, such as a buffer(s), a diluent(s), a standard(s), and/or any other material useful in sample processing, washing, or conducting any other step of the assay.

Kits preferably include instructions for carrying out one or more of the screening methods described herein. Instructions included in kits can be affixed to packaging material or can be included as a package insert. While the instructions are typically written or printed materials, they are not limited to such. Any medium capable of storing such instructions and communicating them to an end user can be employed. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

### Example 1—Detection of Neisseria gonorrhoeae by the Echo Assay by Gel

### Analysis

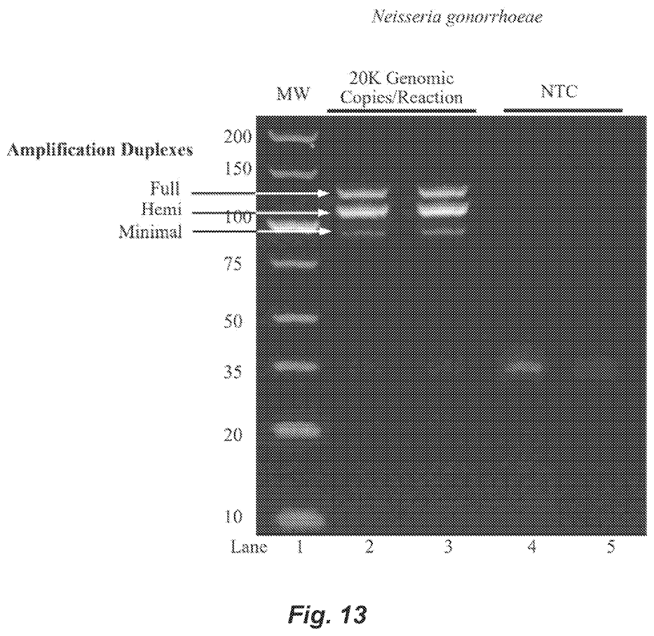

This working example shows the isothermal amplification of Neisseria gonorrhoeae strain FA1090 (ATCC 700825DQ) by Bst 2.0WS (New England Biolabs cat no: M0538M) and RNase H2 (Integrated DNA Technologies cat no: Nov. 3, 2002-03). For FIG. 13, reaction mixtures (20 μL) were prepared on a cold block by mixing corresponding stock solutions for amplification for a final composition of, 8U Bst 2.0WS, 0.8U RNase H2, 1.5 μM linear Fwd primer (5′-GCAGTCGPCCCGPCCGTGrPCTGAATCTTGCGGAAGGTCTGTACGCGCTGA TrGATT-C3-3′, SEQ ID NO:20) and 1.5 μM linear Rev primer (5′-CAPCCCTAPCCGGCGAPCCTCrPCTGCCGCCTATGGTATTGGTAAACGCAAA rCACA-C3-3′, SEQ ID NO:21), 20 mM Tris-HCl (pH 8.0), 40 mM KCl, 9 mM MgCl₂, 0.1% Triton X-100, 0.1 mg/mL BSA, 0.15 mM dA/dG/dTTP, 0.9 mM dCTP, 3 mM rPTP, 0.08 mM dZTP, and 20K copies template or enzyme dilution buffer for NTC reactions. In the sequences disclosed herein, the designation “r” before a base means that the base is a ribobase; “P” refers to the non-natural base 2-amino-8-(10-β-D-2′-deoxyribofuranosyl)-imidazo[1,2-a]-1,3,5-triazin-4(8H)-one; “rP” refers to the non-natural ribobase 2-amino-8-(10-β-D-2′-ribofuranosy])-imidazo[1,2-a]-1,3,5-triazin-4(8H)-one. During preparation of the reaction mixtures, enzymes were always added last. Then the reaction tubes were added to the thermocycler (BioRad C1000) and heated to 70° C. for 20 minutes. Aliquots of the reaction were analyzed with E-Gel EX Agarose Gels 4% (ThermoFisher Scientific). The results of FIG. 13 show that the target was amplified sufficiently for detection after 20 minutes. Lanes 2 and 3 show the anticipated three molecular species: the Full, Hemi and Minimal amplification duplexes. The Hemi amplification duplexes were in greater abundance than the Full and Minimal duplexes and attributed to both operative amplification pathways (Forward and Reverse Hemi amplification duplexes). No off target or primer-dimers were observed in the NTC reactions (Lanes 4 and 5).

### Example 2—Detection of Streptococcus pyogenes Group a by the Echo Assay in Real-Time

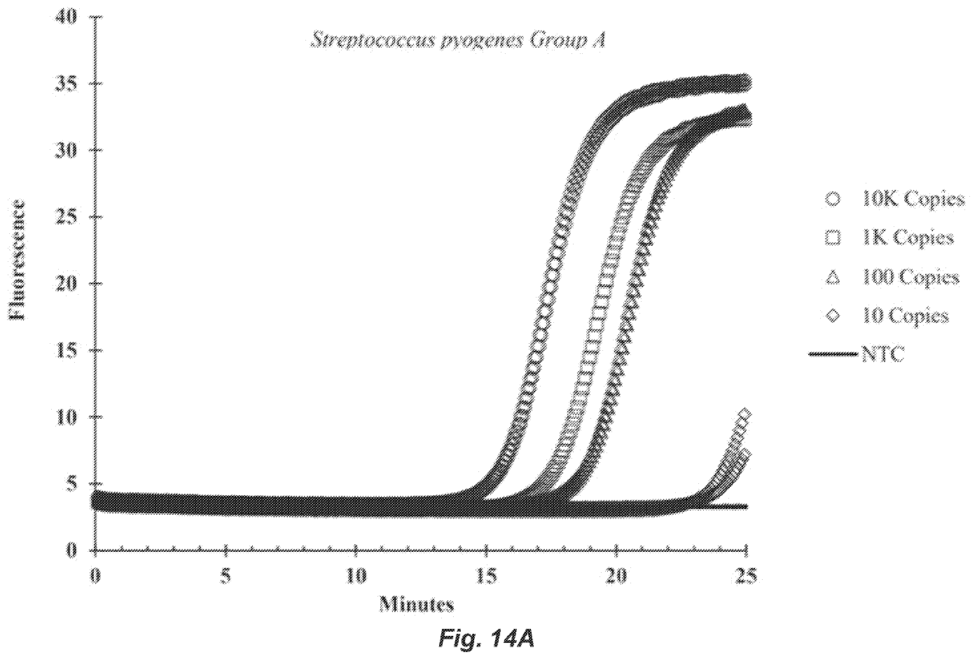

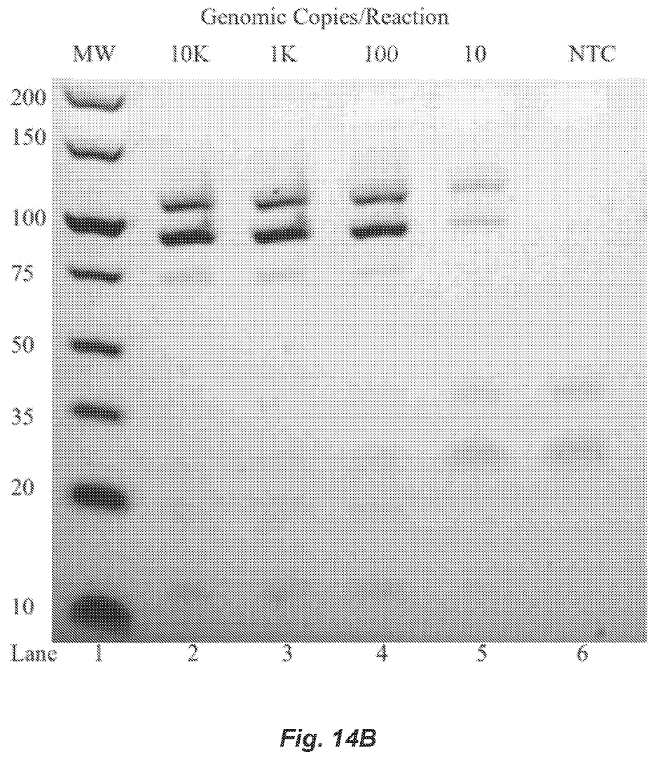

This working example shows the isothermal amplification of Streptococcus pyogenes Group A Strain Bruno (ATCC 19615DQ) by Bst 2.0WS (New England Biolabs cat no: M0538M) and RNase H2 (Integrated DNA Technologies cat no: Nov. 3, 2002-03). For FIG. 14A, reaction mixtures (20 μL) were prepared on a cold block by mixing corresponding stock solutions for amplification for a final composition of, 8U Bst 2.0WS, 0.8U RNase H2, 1.5 μM linear Fwd primer (5′-CACCPCACCTPATCCPAGAGrPGACGGCTTCTTCCGTCTTGACGCA CTrAAAC-C3-3′, SEQ ID NO:22) and 1.5 μM linear Rev primer (5′-AGTAPCACGAPAAGGPTCCCrPAAGCCGCCTGCGCCGCCrACCA-C3-3′, SEQ ID NO: 23), 0.3 μM cycling probe, 0.19 μM strand displacement initiation primer (5′-CCATACTAAGGTTTGATGCCTACAACArGCAC-C3-3′; SEQ ID NO:24), 20 mM Tris-HCl (pH 8.0), 70 mM KCl, 9 mM MgCl₂, 0.1% Triton X-100, 0.1 mg/mL BSA, 0.15 mM dA/dG/dTTP, 0.9 mM dCTP, 3 mM rPTP, 0.08 mM dZTP, enzyme dilution buffer for NTC reactions. The template was added from a 10-fold dilution series for a final copy count per reaction of 10K, 1K, 100 and 10, and tested in duplicate. During preparation of the reaction mixtures, enzymes were always added last. Then the reaction tubes were added to the thermocycler (Rotor-Gene Q, Qiagen) and incubated at 70° C. for 25 minutes. The time (minutes) from the fluorescence profiles shown in FIG. 14A were calculated from threshold crossings and listed in Table 1. Aliquots of the reactions were analyzed for end product analysis with E-Gel EX Agarose Gels 4% (ThermoFisher Scientific). The results of FIG. 14B show that the target is effectively amplified across the entire input range. Lanes 2-5 show products formed from 10K to 10 copies per reaction, and all three molecular species (Full, Hemi, and Minimal amplification duplexes) were formed. No off target or primer-dimers were observed in the NTC reactions (Lanes 4 and 5).

TABLE 1

| Genomic Copies/Reaction | Min. ± SD |
|---|---|
| 10K | 15.0 ± 0.07 |
| 1K | 16.8 ± 0.14 |
| 100 | 18.1 ± 0.00 |
| 10 | 23.6 ± 0.42 |
| 0 | 0 |

### Example 3—Detection of Bacillus subtilis Strain 168 by the Echo Assay in Real-Time

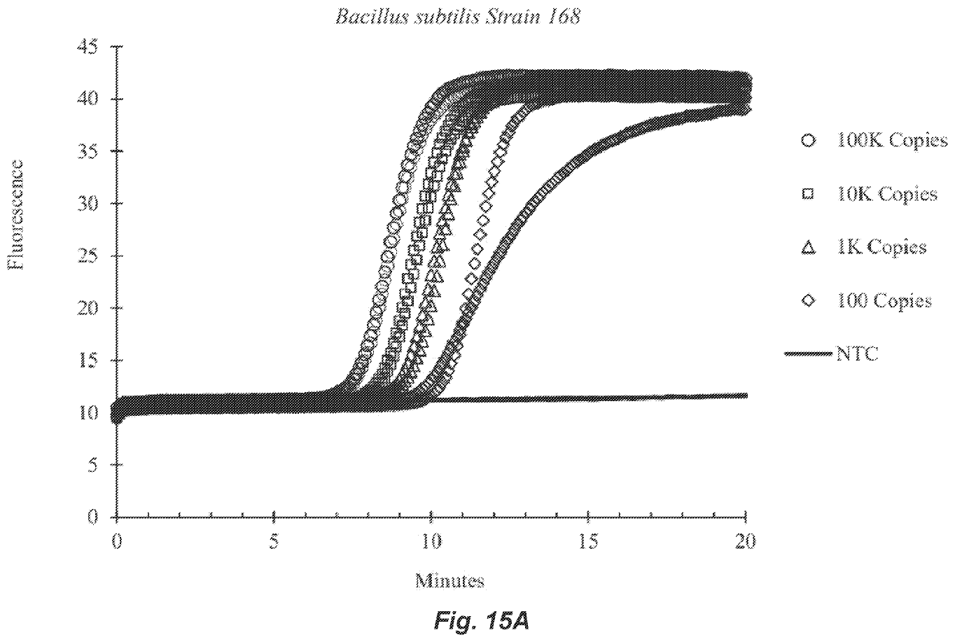

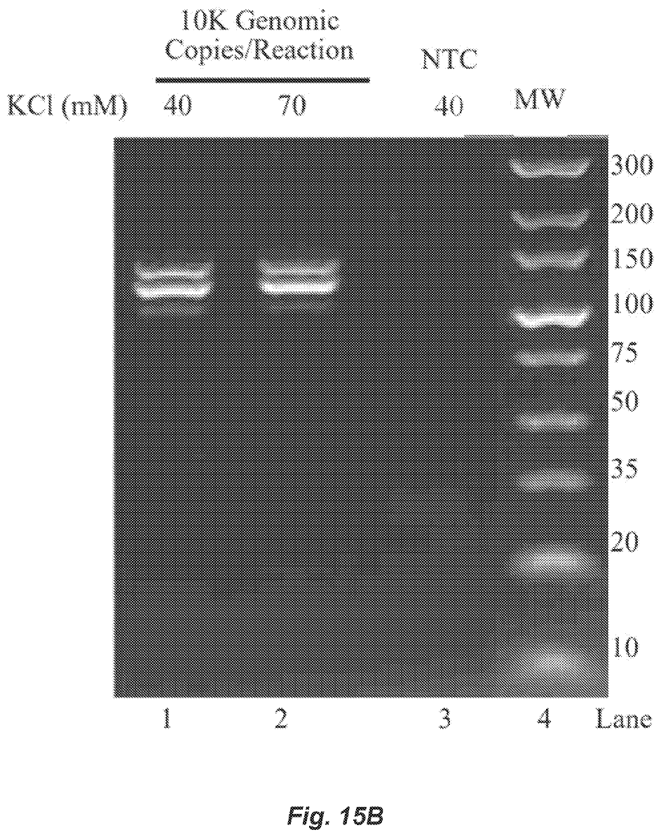

This working example shows the isothermal amplification of Bacillus subtilis Strain 168 (ATCC 23857D-5) by Bst 2.0WS (New England Biolabs cat no: M0538M) and RNase H2 (Integrated DNA Technologies cat no: Nov. 3, 2002-03). For FIG. 15A, reaction mixtures (20 μL) were prepared on a cold block by mixing corresponding stock solutions for amplification for a final composition of, 16U Bst 2.0WS, 0.8U RNase H2, 1.5 μM linear Fwd and Rev primers having the same general structure as those in Examples 1 and 2, 0.45 μM cycling probe, 0.19 μM strand displacement initiation primer having the same general structure as that in Example 2, 20 mM Tris-HCl (pH 8.0), 70 mM KCl, 9 mM MgCl2, 0.1% Triton X-100, 0.1 mg/mL BSA, 0.15 mM dA/dG/dTTP, 0.9 mM dCTP, 3 mM rPTP, 0.08 mM dZTP, enzyme dilution buffer for NTC reactions. The template was added from a 10-fold dilution series for a final copy count per reaction of 100K, 10K, 1K, and 100, and tested in duplicate. During preparation of the reaction mixtures, enzymes were always added last. Then the reaction tubes were added to the thermocycler (Rotor-Gene Q, Qiagen) and incubated at 70° C. for 20 minutes. The time (minutes) from the fluorescence profiles shown in FIG. 15A were calculated from threshold crossings and listed in Table 2. Aliquots from separate reactions, utilizing the same formulation described above with the addition of a 40 mM KCl condition, and an additional 5 minutes of incubation time was added, were analyzed for end product analysis with E-Gel EX Agarose Gels 4% (ThermoFisher Scientific). The results of FIG. 15B show that 10K copies are effectively amplified similarly at both 40 mM and 70 mM KCl (Lanes 1 and 2), contain the anticipated products (Full, Hemi, and Minimal amplification duplexes), and no off target or primer-dimers were observed in the NTC reaction (Lane 3).

TABLE 2

| Genomic Copies/Reaction | Min. ± SD |
|---|---|
| 100K | 7.8 ± 0.0 |
| 10K | 8.6 ± 0.05 |
| 1K | 9.4 ± 0.18 |
| 100 | 10.4 ± 0.22 |
| 0 | 0 |

### Example 4—Detection of Chlamydia trachomatis Serovar D by the Echo Assay in Real-Time

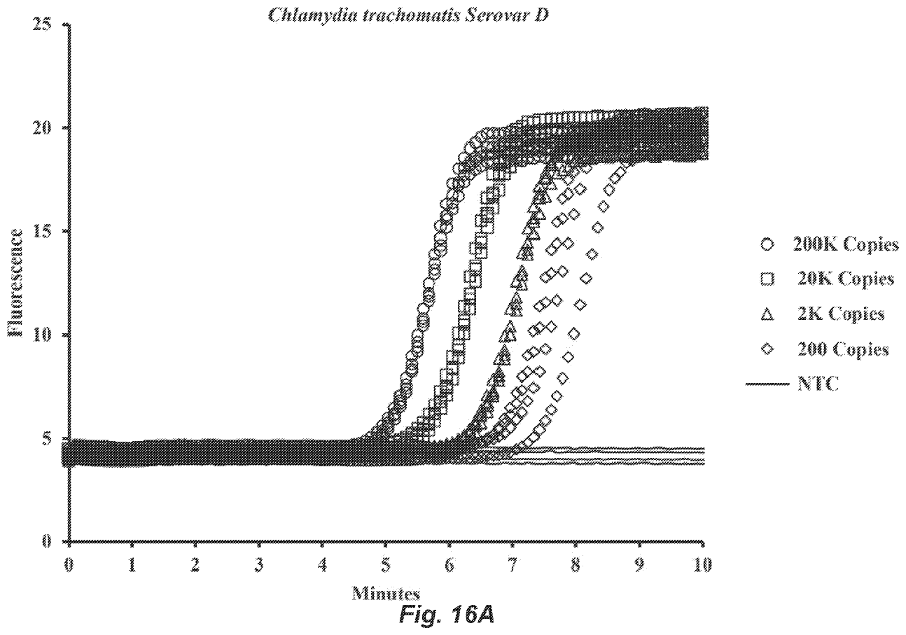

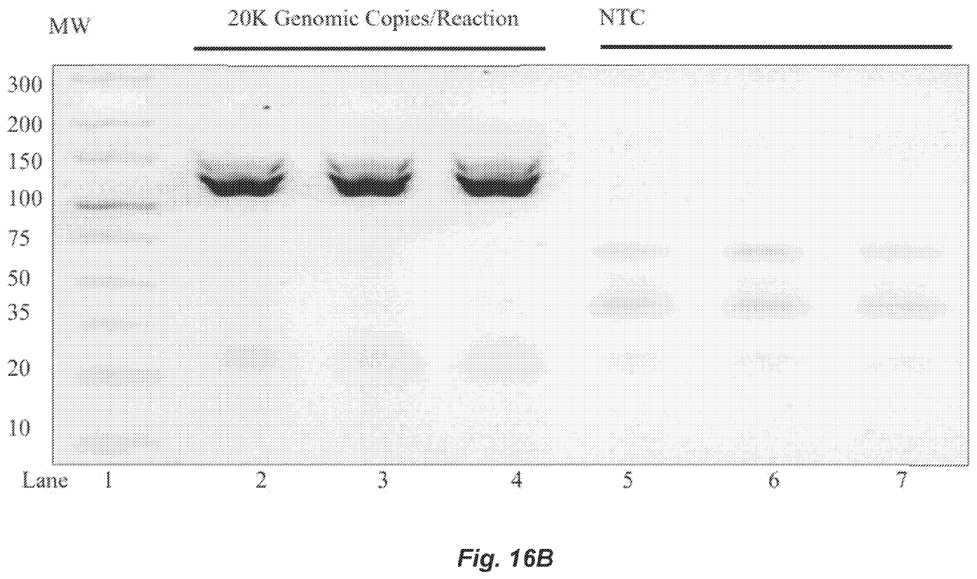

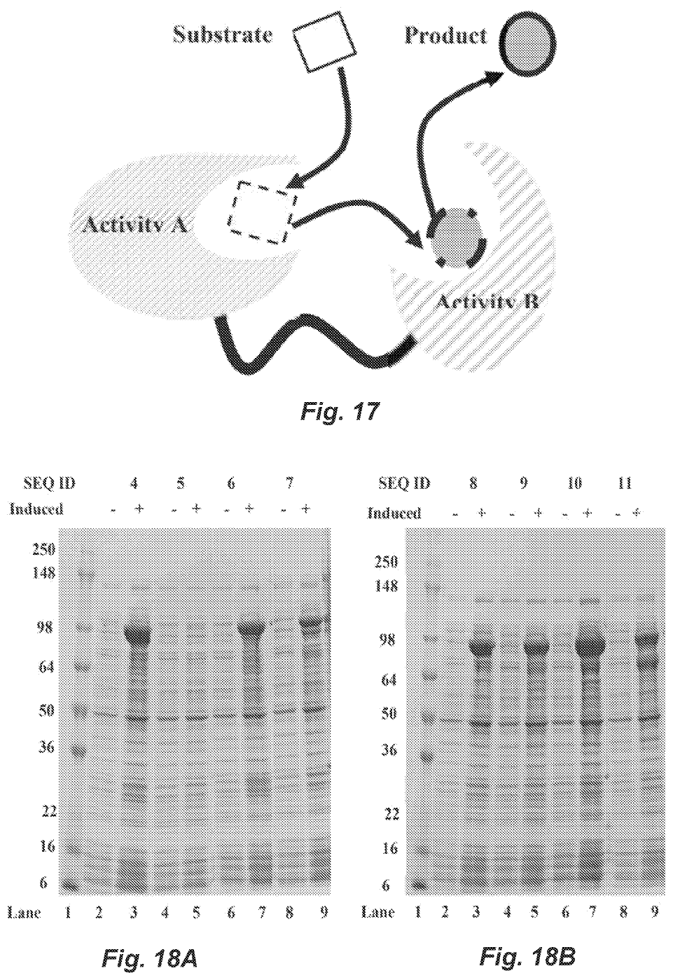

This working example shows the isothermal amplification of Chlamydia trachomatis Serovar D strain UW-3/Cx (ATCC VR-885D) by Bst 2.0WS (New England Biolabs cat no: M0538M) and RNase H2 (Integrated DNA Technologies cat no: Nov. 3, 2002-03). For FIG. 16A, reaction mixtures (20 μL) were prepared on a cold block by mixing corresponding stock solutions for amplification for a final composition of, 16U Bst 2.0WS, 1.6U RNase H2, 1.5 μM linear Fwd and Rev primers having the same general structure as those in Examples 1 and 2, 0.40 μM cycling probe, 0.19 μM strand displacement initiation primer having the same general structure as that in Example 2, 20 mM Tris-HCl (pH 8.0), 70 mM KCl, 9 mM MgCl2, 0.1% Triton X-100, 0.1 mg/mL BSA, 0.15 mM dA/dG/dTTP, 0.9 mM dCTP, 3 mM rPTP, 0.08 mM dZTP, enzyme dilution buffer for NTC reactions. The template was added from a 10-fold dilution series for a final copy count per reaction of 200K, 20K, 2K, and 200, and tested with four replicates. During preparation of the reaction mixtures, enzymes were always added last. Then the reaction tubes were added to the thermocycler (Rotor-Gene Q, Qiagen) and incubated at 70° C. for 20 minutes. The time (minutes) from the fluorescence profiles shown in FIG. 16A were calculated from threshold crossings and listed in Table 3. Aliquots from separate 20 μL reactions, 20K copies template, 8U Bst 2.0WS, 0.8U RNase H2, 1.0 μM Fwd and Rev, 20 mM Tris-HCl (pH 8.0), 70 mM KCl, 9 mM MgCl2, 0.1% Triton X-100, 0.1 mg/mL BSA, 0.15 mM dA/dG/dTTP, 0.9 mM dCTP, 3 mM rPTP, 0.08 mM dZTP, enzyme dilution buffer for NTC reactions, then the reaction tubes (N=3) were added to the thermocycler (BioRad C1000) and incubated at 70° C. for 25 minutes followed by end product analysis with E-Gel EX Agarose Gels 4% (ThermoFisher Scientific). The results of FIG. 16B show that 20K copies were effectively amplified (Lanes 2-4), contain the anticipated products (Full, Hemi, and Minimal amplification duplexes), and no off target or primer-dimers were observed in the NTC reaction (Lanes 5-6).

TABLE 3

| Genomic Copies/Reaction | Min. ± SD |
|---|---|
| 200K | 4.6 ± 0.01 |
| 20K | 5.3 ± 0.01 |
| 2K | 6.0 ± 0.05 |
| 200 | 6.7 ± 0.26 |
| 0 | 0 |

### Example 5—Expression and Purification Chimeras

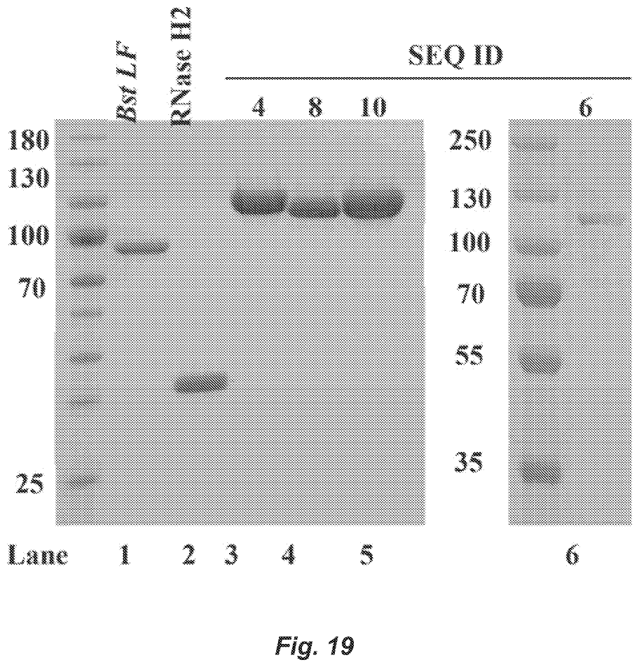

The following steps, which were carried out in this study, illustrate a method for generation of functional fusion proteins between RNase HII and Bst large fragment DNA polymerase.
1. Synthetic genes for SEQ ID NOs: 4-11 were inserted into pET 21(+) expression vectors upstream of the open reading frame (GENEWIZ), and used to transform chemically competent HMS174 (DE3) E. coli cells (Millipore) using the heat shock method with outgrowth in SOC media for 60 minutes at 37° C. Transformed cells were plated on LB agar plates supplemented with 75 μg/mL ampicillin, and grown overnight at 37° C. Transformed colonies were used to inoculate 50 mL 2YT media (Teknova) supplemented with 75 μg/mL carbenicillin, and cultured overnight at 37° C. Overnight cultures were diluted 1:200 with terrific broth (Teknova) containing 100 μg/mL carbenicillin and cultured in shake flask at 37° C. until the OD600 reached 0.8-1.2. Induction performed with 1 mM IPTG for 2-3 hours at 37° C., and subsequently harvested at 10,000x g at 4° C. and stored at −80° C. until needed. Aliquots of induced and non-induced fractions were compared (FIGS. 18A and 18B) by mixing supernatant with an equal volume of SDS sample buffer and analyzed by polyacrylamide gel electrophoresis (SDS-PAGE). Only cell extracts from IPTG-induced cultures showed a prominent protein band apart from SEQ ID NO: 5.
2. Cell pastes were resuspended in lysis buffer (20 mM Tris-Cl pH 7.0, 50 mM NaCl, 10% glycerol, 4 mM EDTA, 1 mM DTT, complete protease inhibitor (Roche), mechanical cell disruption performed with two passes at 30 kPSI (Constant Systems). Lysate was clarified at 4,500 RPM for 60 minutes at 4° C. and Triton X-100 added to a final concentration of 0.01%.
3. Cleared protein extracts were loaded onto a prepared Q Sepharose (Cytiva) column using buffer A (20 mM Tris Cl, pH 7.0, 50 mM NaCl, 1 mM DTT, 10% glycerol, 0.01% TX 100). Proteins were eluted with buffer B (buffer A plus 1M NaCl) over 5 to 10 column volumes. Solution fractions containing the protein of interest were adjusted by dilution or buffer exchanged to ≤10 mS/cm and applied to CM Sepharose (Cytiva) using buffer A for binding and washing steps. The fusions were eluted with buffer B over 5 to 8 column volumes. Eluate fractions containing the target protein were diluted, or buffer exchanged to buffer A conditions and applied to a Heparin column (Cytiva). Fusion enzymes were eluted with buffer B over 5-8 column volumes. Heparin purified fractions were exchanged into buffer C (10 mM Tris-Cl pH 7.5, 100 mM NaCl, 1 mM EDTA, 1 mM DTT, 0.1% TX 100, 10% glycerol) and polished by gel filtration (Superdex 200 Increase, Cytiva). Homogeneity of the enzyme preparations was not less the 95% according to SDS PAGE electrophoresis data on a 4-20% polyacrylamide gel, FIG. 19. The purified fusion enzymes were concentrated, buffer exchanged or dialyzed into storage buffer (buffer C plus 10% or 50% glycerol) and stored at −80° C. or −20° C. respectively.

### Example 6—Detection of Endoribonuclease Activity in Real-Time-Activity A

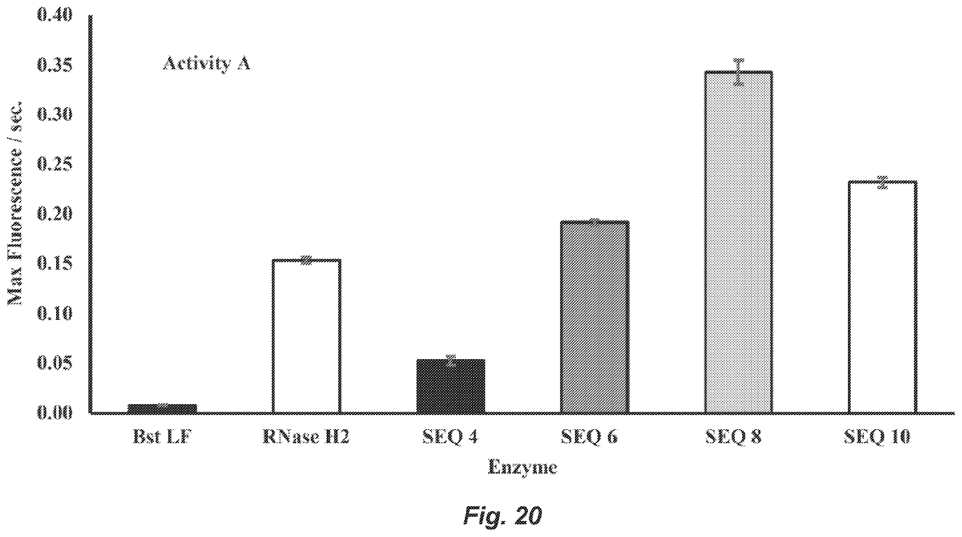

To quantify the catalytic activity of the endonuclease domain, a RNase H2 activity assay was performed. Previously, volume activity was assigned to for each fusion (SEQ ID NOs: 4, 6, 8, and 10) using a standard curve generated with Bst LF (NEB) and reaction conditions described below for polymerase activity (Activity B). Reactions were performed in a 20 μL reaction mixture of 40 mM Tris-Cl pH 8.4, 100 mM KCl, 6 mM MgCl2, 10% glycerol, and 0.02% Brij 58 at 65° C. for 14 minutes. Activity substrate, SEQ ID NO:14, was used at 400 nM. Fusion enzymes were assayed at 5 mU/μL. Positive activity on the substrate requires cleavage 5′ of a single embedded ribonucleotide releasing a fragment containing FAM where fluorescence can be monitored overtime. Reactions were run without enzyme as negative controls for background subtraction. Bst LF (NEB), 5 mU/μL, and RNase H2 (IDT), 0.1 mU/μL were used as negative and positive controls respectively. FIG. 20 shows maximum rates of RNase H2 on SEQ ID NO:14 substrate determined from average maximum slopes of real-time reaction traces. Activities were calculated as the slope (Fluorescence/s). Error bars show standard deviations of four replicates. Enzymes with RNase H2 fused to the C-terminus (SEQ ID NOs: 8 and 10) of Bst LF had greater cleavage activity.

### Example 7—Detection of Polymerase Activity in Real-Time-Activity B

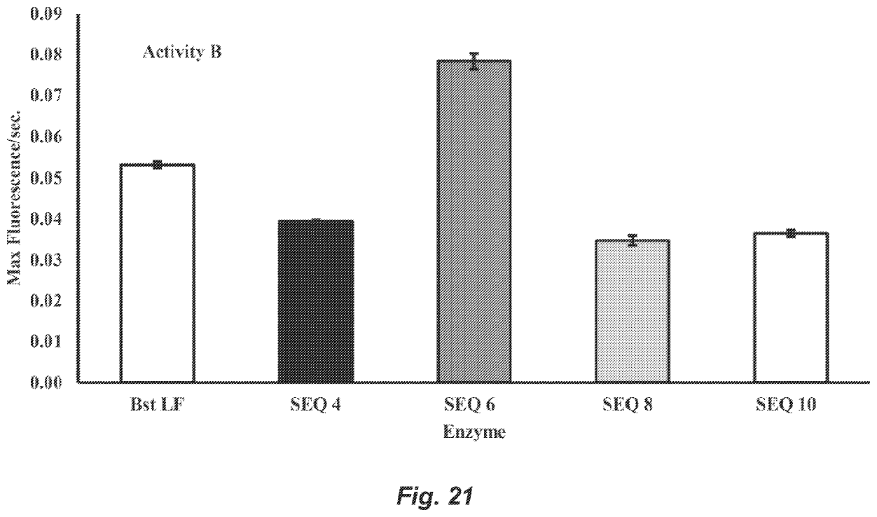

The fusion enzymes (SEQ ID NOs: 4, 6, 8 and 10) were tested for polymerase activity, and assayed via fluorescence monitoring as extension opens the hairpin substrate (SEQ ID NO:15) decoupling the FAM-Dabcyl pair. Reactions were performed in a 20 μL reaction mixture of 50 mM Tris-Cl pH 8.0, 1 mM dNTPs, 6 mM MgCl2, 0.3 μM substrate, 0.1% Tween 20, 100 mM KCl, 1 mM DTT, 0.1 mg/mL BSA. Fusion proteins were tested at 5 mU/μL at 65° C. for 18 minutes. Volume activity, of the fusion enzymes, were assigned based on the polymerase function using a standard curve generated with Bst LF (NEB). Reactions were run without enzyme as negative controls for background subtraction, and Bst LF (NEB) at 5 mU/μL as a positive control. FIG. 21 shows polymerase activity determined from average maximum slopes of real-time reaction traces. Activities were calculated as the slope (Fluorescence/s). Error bars show standard deviations of four replicates. Configurations with RNase H2 fused to the N-terminus (SEQ ID NOs: 4 and 6) of Bst LF exhibited faster extension rates than versions with RNase H2 fused to the C-terminus. SEQ ID NO:6 fused to the N-terminus of Bst LF with the rigid linker (SEQ ID NO: 13) allowed faster extension rates.

### Example 8—Detection of Concerted Catalytic Functions in Real-Time-Activity A+B

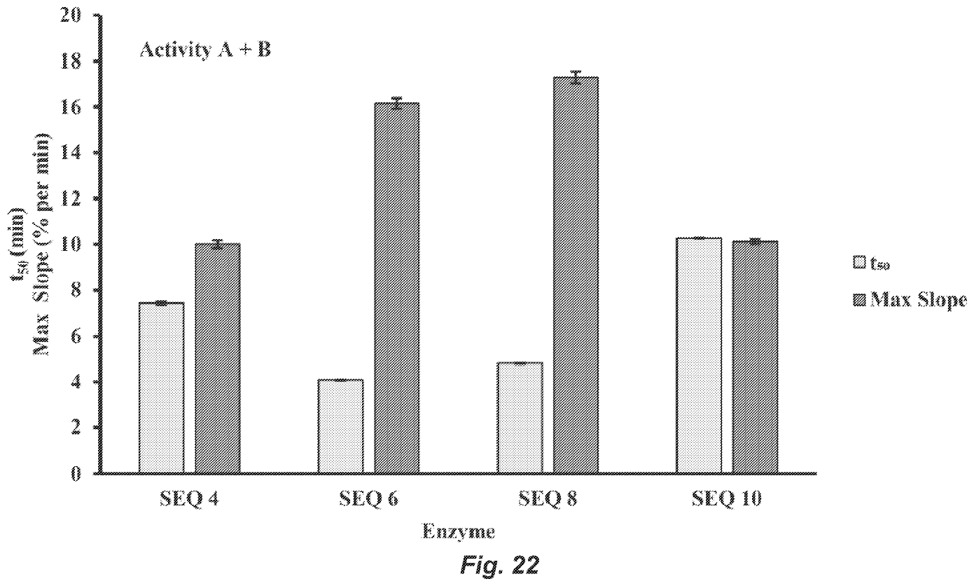

The fusion enzymes (SEQ ID NOs: 4, 6, 8 and 10) were tested for concerted endonuclease and polymerase activity. Reactions were assayed via fluorescence monitoring as activation (3′ unblocking) and extension opens the hairpin substrate (SEQ ID NO:16) decoupling the FAM-Dabcyl pair. Reactions were performed in a 20 μL reaction mixture of 50 mM Tris-Cl pH 8.0, 1 mM dNTPs, 6 mM MgCl2, 1.0 μM substrate (SEQ ID NO:16), 0.1% Tween 20, 100 mM KCl, 1 mM DTT, 0.1 mg/mL BSA. Chimeras were tested at 5 mU/μL at 65° C. for 18 minutes. Reactions were run without enzyme as negative controls for background subtraction. FIG. 22 shows the results of the concerted activity determined from average maximum slopes from normalized real-time reaction traces. Activities were calculated as the slope (%/minute), dark grey bars. The half-life, t₅₀ (minutes) was used to measure the induced response halfway between the minimum and maximum over the reaction duration due to the initial delay phase observed for SEQ ID NO:8 and SEQ ID NO:10. Error bars show standard deviations of four replicates. Two configurations, SEQ ID NO:6 and SEQ ID NO:8 displayed faster overall performance, lower t₅₀ and higher maximum slopes in comparison to SEQ ID NO:4 and SEQ ID NO:10.

### Example 9—Kinetics of Concerted Activation and Extension in Separate Enzyme System Vs One Enzyme System

This working example shows the kinetics of activation and extension by SEQ ID NO:6 chimera configured with RNase H2 fused to the N-terminus of Bst LF through a rigid linker (SEQ ID NO:13). Reactions were assayed via fluorescence monitoring using a linear 3′ capped primer extension assay. As listed in FIG. 23, the template is labelled 5′ with a quencher, and a reporter complementary the 5′ of the template labelled on 3′-end with FAM. The assay requires primer unblocking by RNase H2 leaving a free 3′hydroxyl for Bst LF extension and displacement of the downstream reporter resulting in fluorescence that is monitored in real-time. Briefly, the assay substrate was annealed 1:1:1 (Primer, template, and reporter). Enzyme concentrations were determined using absorbance at 280 nm and extinction coefficients were derived using the Expasy ProtParam tool (Bst LF 54,320 L/mol cm⁻¹, RNase H2 26,930 L/mol cm⁻¹, and SEQ ID NO:6 82,740 L/mol cm⁻¹). Enzymes were assayed at 2.8 nM for kinetic comparison. Briefly, reactions were performed in a 20 μL reaction mixture of 50 mM Tris-Cl pH 8.0, 1 mM dNTPs, 6 mM MgCl2, 0.3 μM annealed substrate, 0.1% Tween 20, 100 mM KCl, 1 mM DTT, 0.1 mg/mL BSA. Reactions were run at 65° C. and monitored in real-time for 18 minutes. Reactions were run without enzyme as negative controls for background subtraction. Activities were calculated as the average maximum slope (fluorescence/s) from the real-time traces. Error bars show standard deviations of four replicates. The one-enzyme system, fused Bst LF with RNase H2 (SEQ ID NO:6) was 1.8x faster relative to the two-enzyme system where Bst LF and RNase H2 were added individually.

### REFERENCES

1. Patel, P. H., et. al. J. Biol. Chem., 2000, 275, 40266-40272.
2. Ibach, J., et. al. J. Biotechnol., 2013, 167, 287-295. 4715.
3. Summerer, D., et. al. Angew. Chem., Int. Ed., 2005, 44, 4712-
4. Oscorbin, I. P., et. al, doi: https://doi.org/10.1101/2020.07.02.185637.
5. Povilaitis, T., et. al. Protein Eng., Des. Sel., 2016, 29, 617-628.

SEQUENCES
Bacillus stearothermophilus DNA polymerase I (polA) Full Length 876 amino acids
SEQ ID NO: 1:
MKKKLVLIDGNSVAYRAFFALPLLHNDKGIHTNAVYGFTMMLNKILAEEQPTHLLVAFDAGKTTFRHETFQEYKGGRQQTPPELSEQF
PLLRELLKAYRIPAYELDHYEADDIIGTLAARAEQEGFEVKIISGDRDLTQLASRHVTVDITKKGITDIEPYTPETVREKYGLTPEQI
VDLKGLMGDKSDNIPGVPGIGEKTAVKLLKQFGTVENVLASIDEVKGEKLKENLRQHRDLALLSKQLASICRDAPVELSLDDIVYEGQ
DREKVIALFKELGFQSFLEKMAAPAAEGEKPLEEMEFAIVDVITEEMLADKAALVVEVMEENYHDAPIVGIALVNEHGRFFMRPETAL
ADSQFLAWLADETKKKSMFDAKRAVVALKWKGIELRGVAFDLLLAAYLLNPAQDAGDIAAVAKMKQYEAVRSDEAVYGKGVKRSLPDE
QTLAEHLVRKAAAIWALEQPFMDDLRNNEQDQLLTKLEQPLAAILAEMEFTGVNVDTKRLEQMGSELAEQLRAIEQRIYELAGQEFNI
NSPKQLGVILFEKLQLPVLKKTKTGYSTSADVLEKLAPHHEIVENILHYRQLGKLQSTYIEGLLKVVRPDTGKVHTMFNQALTQTGRL
SSAEPNLQNIPIRLEEGRKIRQAFVPSEPDWLIFAADYSQIELRVLAHIADDDNLIEAFQRDLDIHTKTAMDIFHVSEEEVTANMRRQ
AKAVNFGIVYGISDYGLAQNLNITRKEAAEFIERYFASFPGVKQYMENIVQEAKQKGYVTTLLHRRRYLPDITSRNFNVRSFAERTAM
NTPIQGSAADIIKKAMIDLAARLKEEQLQARLLLQVHDELILEAPKEEIERLCELVPEVMEQAVTLRVPLKVDYHYGPTWYDAK
Bacillus stearothermophilus DNA polymerase I (polA) Large Fragment 587 amino acids
SEQ ID NO: 2:
AEGEKPLEEMEFAIVDVITEEMLADKAALVVEVMEENYHDAPIVGIALVNEHGRFFMRPETALADSQFLAWLADETKKKSMFDAKRAV
VALKWKGIELRGVAFDLLLAAYLLNPAQDAGDIAAVAKMKQYEAVRSDEAVYGKGVKRSLPDEQTLAEHLVRKAAAIWALEQPFMDDL
RNNEQDQLLTKLEQPLAAILAEMEFTGVNVDTKRLEQMGSELAEQLRAIEQRIYELAGQEFNINSPKQLGVILFEKLQLPVLKKTKTG
YSTSADVLEKLAPHHEIVENILHYRQLGKLQSTYIEGLLKVVRPDTGKVHTMFNQALTQTGRLSSAEPNLQNIPIRLEEGRKIRQAFV
PSEPDWLIFAADYSQIELRVLAHIADDDNLIEAFQRDLDIHTKTAMDIFHVSEEEVTANMRRQAKAVNFGIVYGISDYGLAQNLNITR
KEAAEFIERYFASFPGVKQYMENIVQEAKQKGYVTTLLHRRRYLPDITSRNFNVRSFAERTAMNTPIQGSAADIIKKAMIDLAARLKE
EQLQARLLLQVHDELILEAPKEEIERLCELVPEVMEQAVTLRVPLKVDYHYGPTWYDAK
rnhB ribonuclease HII Pyrococcus abyssi GE5 223 amino acids
SEQ ID NO: 3:
KVAGADEAGRGPVIGPLVIVAAVVEEDKIRSLTKLGVKDSKQLTPAQREKLFDEIVKVLDDYSVVIVSPQDIDGRKGSMNELEVENFV
KALNSLKVKPEVIYIDSADVKAERFAENIRSRLAYEAKVVAEHKADAKYEIVSAASILAKVIRDREIEKLKAEYGDFGSGYPSDPRTK
KWLEEWYSKHGNFPPIVRRTWDTAKKIEEKFKRAQLTLDNFLKRFRN
Chimera His-TEV-RNase H2-FI1a-Bst LF (Clone 6)
SEQ ID NO: 4:
HHHHHHENLYFQGMKVAGADEAGRGPVIGPLVIVAAVVEEDKIRSLTKLGVKDSKQLTPAQREKLFDEIVKVLDDYSVVIVSPQDIDG
RKGSMNELEVENFVKALNSLKVKPEVIYIDSADVKAERFAENIRSRLAYEAKVVAEHKADAKYEIVSAASILAKVIRDREIEKLKAEY
GDFGSGYPSDPRTKKWLEEWYSKHGNFPPIVRRTWDTAKKIEEKFKRAQLTLDNFLKRFRNGTGSGAAEGEKPLEEMEFAIVDVITEE
MLADKAALVVEVMEENYHDAPIVGIALVNEHGRFFMRPETALADSQFLAWLADETKKKSMFDAKRAVVALKWKGIELRGVAFDLLLAA
YLLNPAQDAGDIAAVAKMKQYEAVRSDEAVYGKGVKRSLPDEQTLAEHLVRKAAAIWALEQPFMDDLRNNEQDQLLTKLEQPLAAILA
EMEFTGVNVDTKRLEQMGSELAEQLRAIEQRIYELAGQEFNINSPKQLGVILFEKLQLPVLKKTKTGYSTSADVLEKLAPHHEIVENI
LHYRQLGKLQSTYIEGLLKVVRPDTGKVHTMFNQALTQTGRLSSAEPNLQNIPIRLEEGRKIRQAFVPSEPDWLIFAADYSQIELRVL
AHIADDDNLIEAFQRDLDIHTKTAMDIFHVSEEEVTANMRRQAKAVNFGIVYGISDYGLAQNLNITRKEAAEFIERYFASFPGVKQYM
ENIVQEAKQKGYVTTLLHRRRYLPDITSRNFNVRSFAERTAMNTPIQGSAADIIKKAMIDLAARLKEEQLQARLLLQVHDELILEAPK
EEIERLCELVPEVMEQAVTLRVPLKVDYHYGPTWYDAK
Chimera RNase H2-FI1a-Bst LF-TEV-His (Clone 7)
SEQ ID NO: 5:
KVAGADEAGRGPVIGPLVIVAAVVEEDKIRSLTKLGVKDSKQLTPAQREKLFDEIVKVLDDYSVVIVSPQDIDGRKGSMNELEVENFV
KALNSLKVKPEVIYIDSADVKAERFAENIRSRLAYEAKVVAEHKADAKYEIVSAASILAKVIRDREIEKLKAEYGDFGSGYPSDPRTK
KWLEEWYSKHGNFPPIVRRTWDTAKKIEEKFKRAQLTLDNFLKRFRNGTGSGAAEGEKPLEEMEFAIVDVITEEMLADKAALVVEVME
ENYHDAPIVGIALVNEHGRFFMRPETALADSQFLAWLADETKKKSMFDAKRAVVALKWKGIELRGVAFDLLLAAYLLNPAQDAGDIAA
VAKMKQYEAVRSDEAVYGKGVKRSLPDEQTLAEHLVRKAAAIWALEQPFMDDLRNNEQDQLLTKLEQPLAAILAEMEFTGVNVDTKRL
EQMGSELAEQLRAIEQRIYELAGQEFNINSPKQLGVILFEKLQLPVLKKTKTGYSTSADVLEKLAPHHEIVENILHYRQLGKLQSTYI
EGLLKVVRPDTGKVHTMFNQALTQTGRLSSAEPNLQNIPIRLEEGRKIRQAFVPSEPDWLIFAADYSQIELRVLAHIADDDNLIEAFQ
RDLDIHTKTAMDIFHVSEEEVTANMRRQAKAVNFGIVYGISDYGLAQNLNITRKEAAEFIERYFASFPGVKQYMENIVQEAKQKGYVT
TLLHRRRYLPDITSRNFNVRSFAERTAMNTPIQGSAADIIKKAMIDLAARLKEEQLQARLLLQVHDELILEAPKEEIERLCELVPEVM
EQAVTLRVPLKVDYHYGPTWYDAKENLYFQGHHHHHH
Chimera His-TEV-RNase H2-HL2a-Bst LF (Clone 8)
SEQ ID NO: 6:
HHHHHHENLYFQGMKVAGADEAGRGPVIGPLVIVAAVVEEDKIRSLTKLGVKDSKQLTPAQREKLFDEIVKVLDDYSVVIVSPQDIDG
RKGSMNELEVENFVKALNSLKVKPEVIYIDSADVKAERFAENIRSRLAYEAKVVAEHKADAKYEIVSAASILAKVIRDREIEKLKAEY
GDFGSGYPSDPRTKKWLEEWYSKHGNFPPIVRRTWDTAKKIEEKFKRAQLTLDNFLKRFRNLAEAAAKEAAAKEAAAKEAAAKEAAAK
AAAAEGEKPLEEMEFAIVDVITEEMLADKAALVVEVMEENYHDAPIVGIALVNEHGRFFMRPETALADSQFLAWLADETKKKSMFDAK
RAVVALKWKGIELRGVAFDLLLAAYLLNPAQDAGDIAAVAKMKQYEAVRSDEAVYGKGVKRSLPDEQTLAEHLVRKAAAIWALEQPFM
DDLRNNEQDQLLTKLEQPLAAILAEMEFTGVNVDTKRLEQMGSELAEQLRAIEQRIYELAGQEFNINSPKQLGVILFEKLQLPVLKKT
KTGYSTSADVLEKLAPHHEIVENILHYRQLGKLQSTYIEGLLKVVRPDTGKVHTMFNQALTQTGRLSSAEPNLQNIPIRLEEGRKIRQ
AFVPSEPDWLIFAADYSQIELRVLAHIADDDNLIEAFQRDLDIHTKTAMDIFHVSEEEVTANMRRQAKAVNFGIVYGISDYGLAQNLN
ITRKEAAEFIERYFASFPGVKQYMENIVQEAKQKGYVTTLLHRRRYLPDITSRNFNVRSFAERTAMNTPIQGSAADIIKKAMIDLAAR
LKEEQLQARLLLQVHDELILEAPKEEIERLCELVPEVMEQAVTLRVPLKVDYHYGPTWYDAK
Chimera RNase H2-HL2a-Bst LF-TEV-His (Clone 9)
SEQ ID NO: 7:
KVAGADEAGRGPVIGPLVIVAAVVEEDKIRSLTKLGVKDSKQLTPAQREKLFDEIVKVLDDYSVVIVSPQDIDGRKGSMNELEVENFV
KALNSLKVKPEVIYIDSADVKAERFAENIRSRLAYEAKVVAEHKADAKYEIVSAASILAKVIRDREIEKLKAEYGDFGSGYPSDPRTK
KWLEEWYSKHGNFPPIVRRTWDTAKKIEEKFKRAQLTLDNFLKRFRNLAEAAAKEAAAKEAAAKEAAAKEAAAKAAAAEGEKPLEEME
FAIVDVITEEMLADKAALVVEVMEENYHDAPIVGIALVNEHGRFFMRPETALADSQFLAWLADETKKKSMFDAKRAVVALKWKGIELR
GVAFDLLLAAYLLNPAQDAGDIAAVAKMKQYEAVRSDEAVYGKGVKRSLPDEQTLAEHLVRKAAAIWALEQPFMDDLRNNEQDQLLTK
LEQPLAAILAEMEFTGVNVDTKRLEQMGSELAEQLRAIEQRIYELAGQEFNINSPKQLGVILFEKLQLPVLKKTKTGYSTSADVLEKL
APHHEIVENILHYRQLGKLQSTYIEGLLKVVRPDTGKVHTMFNQALTQTGRLSSAEPNLQNIPIRLEEGRKIRQAFVPSEPDWLIFAA
DYSQIELRVLAHIADDDNLIEAFQRDLDIHTKTAMDIFHVSEEEVTANMRRQAKAVNFGIVYGISDYGLAQNLNITRKEAAEFIERYF
ASFPGVKQYMENIVQEAKQKGYVTTLLHRRRYLPDITSRNFNVRSFAERTAMNTPIQGSAADIIKKAMIDLAARLKEEQLQARLLLQV
HDELILEAPKEEIERLCELVPEVMEQAVTLRVPLKVDYHYGPTWYDAKENLYFQGHHHHHH
Chimera His-TEV-Bst LF-FL1a-RNase H2 (Clone 10)
SEQ ID NO: 8:
HHHHHHENLYFQGAEGEKPLEEMEFAIVDVITEEMLADKAALVVEVMEENYHDAPIVGIALVNEHGRFFMRPETALADSQFLAWLADE
TKKKSMFDAKRAVVALKWKGIELRGVAFDLLLAAYLLNPAQDAGDIAAVAKMKQYEAVRSDEAVYGKGVKRSLPDEQTLAEHLVRKAA
AIWALEQPFMDDLRNNEQDQLLTKLEQPLAAILAEMEFTGVNVDTKRLEQMGSELAEQLRAIEQRIYELAGQEFNINSPKQLGVILFE
KLQLPVLKKTKTGYSTSADVLEKLAPHHEIVENILHYRQLGKLQSTYIEGLLKVVRPDTGKVHTMFNQALTQTGRLSSAEPNLQNIPI
RLEEGRKIRQAFVPSEPDWLIFAADYSQIELRVLAHIADDDNLIEAFQRDLDIHTKTAMDIFHVSEEEVTANMRRQAKAVNFGIVYGI
SDYGLAQNLNITRKEAAEFIERYFASFPGVKQYMENIVQEAKQKGYVTTLLHRRRYLPDITSRNFNVRSFAERTAMNTPIQGSAADII
KKAMIDLAARLKEEQLQARLLLQVHDELILEAPKEEIERLCELVPEVMEQAVTLRVPLKVDYHYGPTWYDAKGTGSGAMKVAGADEAG
RGPVIGPLVIVAAVVEEDKIRSLTKLGVKDSKQLTPAQREKLFDEIVKVLDDYSVVIVSPQDIDGRKGSMNELEVENFVKALNSLKVK
PEVIYIDSADVKAERFAENIRSRLAYEAKVVAEHKADAKYEIVSAASILAKVIRDREIEKLKAEYGDFGSGYPSDPRTKKWLEEWYSK
HGNFPPIVRRTWDTAKKIEEKFKRAQLTLDNFLKRFRN
Chimera Bst LF-FL1a-RNase H2-TEV-His (Clone 11)
SEQ ID NO: 9:
AEGEKPLEEMEFAIVDVITEEMLADKAALVVEVMEENYHDAPIVGIALVNEHGRFFMRPETALADSQFLAWLADETKKKSMFDAKRAV
VALKWKGIELRGVAFDLLLAAYLLNPAQDAGDIAAVAKMKQYEAVRSDEAVYGKGVKRSLPDEQTLAEHLVRKAAAIWALEQPFMDDL
RNNEQDQLLTKLEQPLAAILAEMEFTGVNVDTKRLEQMGSELAEQLRAIEQRIYELAGQEFNINSPKQLGVILFEKLQLPVLKKTKTG
YSTSADVLEKLAPHHEIVENILHYRQLGKLQSTYIEGLLKVVRPDTGKVHTMFNQALTQTGRLSSAEPNLQNIPIRLEEGRKIRQAFV
PSEPDWLIFAADYSQIELRVLAHIADDDNLIEAFQRDLDIHTKTAMDIFHVSEEEVTANMRRQAKAVNFGIVYGISDYGLAQNLNITR
KEAAEFIERYFASFPGVKQYMENIVQEAKQKGYVTTLLHRRRYLPDITSRNFNVRSFAERTAMNTPIQGSAADIIKKAMIDLAARLKE
EQLQARLLLQVHDELILEAPKEEIERLCELVPEVMEQAVTLRVPLKVDYHYGPTWYDAKGTGSGAMKVAGADEAGRGPVIGPLVIVAA
VVEEDKIRSLTKLGVKDSKQLTPAQREKLFDEIVKVLDDYSVVIVSPQDIDGRKGSMNELEVENFVKALNSLKVKPEVIYIDSADVKA
ERFAENIRSRLAYEAKWVAEHKADAKYEIVSAASILAKVIRDREIEKLKAEYGDFGSGYPSDPRTKKWLEEWYSKHGNFPPIVRRTWD
TAKKIEEKFKRAQLTLDNFLKRFRNENLYFQGHHHHHH
Chimera His-TEV-Bst LF-HL2a-RNase H2 (Clone 12)
SEQ ID NO: 10:
HHHHHHENLYFQGAEGEKPLEEMEFAIVDVITEEMLADKAALVVEVMEENYHDAPIVGIALVNEHGRFFMRPETALADSQFLAWLADE
TKKKSMFDAKRAVVALKWKGIELRGVAFDLLLAAYLLNPAQDAGDIAAVAKMKQYEAVRSDEAVYGKGVKRSLPDEQTLAEHLVRKAA
AIWALEQPFMDDLRNNEQDQLLTKLEQPLAAILAEMEFTGVNVDTKRLEQMGSELAEQLRAIEQRIYELAGQEFNINSPKQLGVILFE
KLQLPVLKKTKTGYSTSADVLEKLAPHHEIVENILHYRQLGKLQSTYIEGLLKVVRPDTGKVHTMFNQALTQTGRLSSAEPNLQNIPI
RLEEGRKIRQAFVPSEPDWLIFAADYSQIELRVLAHIADDDNLIEAFQRDLDIHTKTAMDIFHVSEEEVTANMRRQAKAVNFGIVYGI
SDYGLAQNLNITRKEAAEFIERYFASFPGVKQYMENIVQEAKQKGYVTTLLHRRRYLPDITSRNFNVRSFAERTAMNTPIQGSAADII
KKAMIDLAARLKEEQLQARLLLQVHDELILEAPKEEIERLCELVPEVMEQAVTLRVPLKVDYHYGPTWYDAKLAEAAAKEAAAKEAAA
KEAAAKEAAAKAAAMKVAGADEAGRGPVIGPLVIVAAVVEEDKIRSLTKLGVKDSKQLTPAQREKLFDEIVKVLDDYSVVIVSPQDID
GRKGSMNELEVENFVKALNSLKVKPEVIYIDSADVKAERFAENIRSRLAYEAKWVAEHKADAKYEIVSAASILAKVIRDREIEKLKAE
YGDFGSGYPSDPRTKKWLEEWYSKHGNFPPIVRRTWDTAKKIEEKFKRAQLTLDNFLKRFRN
Chimera -Bst LF-HL2a-RNase H2-TEV-His (Clone 13)
SEQ ID NO: 11:
AEGEKPLEEMEFAIVDVITEEMLADKAALVVEVMEENYHDAPIVGIALVNEHGRFFMRPETALADSQFLAWLADETKKKSMFDAKRAV
VALKWKGIELRGVAFDLLLAAYLLNPAQDAGDIAAVAKMKQYEAVRSDEAVYGKGVKRSLPDEQTLAEHLVRKAAAIWALEQPFMDDL
RNNEQDQLLTKLEQPLAAILAEMEFTGVNVDTKRLEQMGSELAEQLRAIEQRIYELAGQEFNINSPKQLGVILFEKLQLPVLKKTKTG
YSTSADVLEKLAPHHEIVENILHYRQLGKLQSTYIEGLLKVVRPDTGKVHTMFNQALTQTGRLSSAEPNLQNIPIRLEEGRKIRQAFV
PSEPDWLIFAADYSQIELRVLAHIADDDNLIEAFQRDLDIHTKTAMDIFHVSEEEVTANMRRQAKAVNFGIVYGISDYGLAQNLNITR
KEAAEFIERYFASFPGVKQYMENIVQEAKQKGYVTTLLHRRRYLPDITSRNFNVRSFAERTAMNTPIQGSAADIIKKAMIDLAARLKE
EQLQARLLLQVHDELILEAPKEEIERLCELVPEVMEQAVTLRVPLKVDYHYGPTWYDAKLAEAAAKEAAAKEAAAKEAAAKEAAAKAA
AMKVAGADEAGRGPVIGPLVIVAAVVEEDKIRSLTKLGVKDSKQLTPAQREKLFDEIVKVLDDYSVVIVSPQDIDGRKGSMNELEVEN
FVKALNSLKVKPEVIYIDSADVKAERFAENIRSRLAYEAKVVAEHKADAKYEIVSAASILAKVIRDREIEKLKAEYGDFGSGYPSDPR
TKKWLEEWYSKHGNFPPIVRRTWDTAKKIEEKFKRAQLTLDNFLKRFRNENLYFQGHHHHHH
Flexible Linker
SEQ ID NO: 12:
GTGSGA
Helical Linker, LA(EAAAK)₅AAA
SEQ ID NO: 13:
LAEAAAKEAAAKEAAAKEAAAKEAAAKAAA
RNase H2 Cleavage Substrate
SEQ ID NO: 14:
5′-CDQ13R-ACTTTGAGACTGGCGCGAAGCGCCAGTCTcAAAGT-FAM-C3
Polymerase Extension Substrate
SEQ ID NO: 15:
5′-FAM-CCGTCGCTGGCGAAGCCAGCGACGGCGTCACTGATTGTGCACAGAGGCGCCTCGAGCGCGAAGCGCTCGAGGCGCC-3′
Endonuclease Cleavage and Polymerase Extension Substrate
SEQ ID NO: 16:
5′-FAM-CGCTCCGTGGCGAAGCCACGGAGCGGGTCACTGATTGTCCACACAGTGTGGTAGAGCCAGCGCGAAGCGCTGGCTCTACCA
CACT-C3-3′
Labelled Template Sequence
SEQ ID NO: 17:
5′-CDQ13R-GCGTAGCTGACTGCAGCTGCAGCGACGGCGTCACTGATTGTGCACAGAGGCGCCTCGAGCGC-3′
Blocked Primer Sequence
SEQ ID NO: 18:
5′-G(+C)G(+C)TCGAGGCGcCTCTG-C3-3′
where +indicates LNA base and lowercase indicates native ribobase
Reporter Sequence
SEQ ID NO: 19:
5′-GCTGCAGCTGCAGTCAGCTACGC(FAM)-C3-3′
Forward Primer, Example 1
SEQ ID NO: 20:
5′-GCAGTCGPCCCGPCCGTGrPCTGAATCTTGCGGAAGGTCTGTACGCGCTGATrGATT-C3-3′
Reverse Primer, Example 1
SEQ ID NO: 21:
5′-CAPCCCTAPCCGGCGAPCCTCrPCTGCCGCCTATGGTATTGGTAAACGCAAArCACA-C3-3′
Forward Primer, Example 2
SEQ ID NO: 22:
5′-CACCPCACCTPATCCPAGAGrPGACGGCTTCTTCCGTCTTGACGCACTrAAAC-C3-3′
Reverse Primer, Example 2
SEQ ID NO: 23:
5′-AGTAPCACGAPAAGGPTCCCrPAAGCCGCCTGCGCCGCCrACCA-C3-3′
Strand Displacement Initiation Primer, Example 2
SEQ ID NO: 24:
5′-CCATACTAAGGTTTGATGCCTACAACArGCAC-C3-3′

What is claimed is:

1. A nucleic acid construct comprising:
a target-specific region; and,
a universal adapter sequence located 5′ of the target-specific region, wherein the universal adapter sequence comprises a non-natural nucleotide base.

2. The nucleic acid construct of claim 1, wherein the nucleic acid construct additionally comprises a terminal 3′ cap.

3. The nucleic acid construct of claim 1 or claim 2, wherein the target-specific region comprises a target-specific cleavage domain.

4. The nucleic acid construct of claim 3, wherein the target-specific cleavage domain comprises a ribonucleotide.

5. The nucleic acid construct of any one of claims 1-4, wherein the non-natural nucleotide base is a ribonucleotide.

6. The nucleic acid construct of any one of claims 1-4, where in the non-natural nucleotide base is a deoxyribonucleotide.

7. The nucleic acid construct of any one of claims 1-4, wherein the non-natural nucleotide base is selected from the group consisting of xanthosine, isoguanosine, deoxyxanthosine, deoxyisoguanosine, isocytosine, deoxyisocytosine, 6-amino-5-nitro-3-(1′-beta-D-2′-ribofuranosyl)-2(1H)-pyridone, 6-amino-5-nitro-3-(1′-beta-D-2′-deoxyribofuranosyl)-2(1H)-pyridone, 2-amino-8- (1′-beta-D-2′-ribofuranosyl)-imidazo[1,2-a]-1,3,5-triazin-4(8H)-one, and 2-amino-8-(1′-beta-D-2′-deoxyribofuranosyl)-imidazo[1,2-a]-1,3,5-triazin-4(8H)-one.

8. The nucleic acid construct of claim 7, wherein the non-natural nucleotide base is selected from the group consisting of 6-amino-5-nitro-3-(1′-beta-D-2′-ribofuranosyl)-2(1H)-pyridone or a 6-amino-5-nitro-3-(1′-beta-D-2′-deoxyribofuranosyl)-2(1H)-pyridone, 2-amino-8-(1′-beta-D-2′-ribofuranosyl)-imidazo[1,2-a]-1,3,5-triazin-4(8H)-one, and 2-amino-8-(1′-beta-D-2′-deoxyribofuranosyl)-imidazo[1,2-a]-1,3,5-triazin-4(8H)-one.

9. The nucleic acid construct of claim 7, wherein the non-natural nucleotide base is a 2-amino-8-(1′-beta-D-2′-ribofuranosyl)-imidazo[1,2-a]-1,3,5-triazin-4(8H)-one or a 2-amino-8-(1′-beta-D-2′-deoxyribofuranosyl)-imidazo[1,2-a]-1,3,5-triazin-4(8H)-one.

10. A fusion protein comprising: a polymerase, or a fragment thereof, and an endonuclease, or a fragment thereof.

11. The fusion protein of claim 10, wherein the polymerase is a Bst DNA polymerase, or a fragment thereof.

12. The fusion protein of claim 11, wherein the polymerase is the large fragment of the Bst DNA polymerase.

13. The fusion protein of claim 11, wherein the polymerase is a Bst 2.0 DNA polymerase, or a fragment thereof.

14. The fusion protein of any one of claims 10-13, wherein the endonuclease is an endoribonuclease.

15. The fusion protein of claim 14, wherein the endoribonuclease is an RNase H2, or a fragment thereof.

16. A primer pair comprising: a forward primer and a reverse primer, the forward and reverse primers each comprising the nucleic acid construct of any one of claims 1-9.

17. The primer pair of claim 16, wherein the forward primer additionally comprises a terminal 3′ cap.

18. The primer pair of claim 16 or claim 17, wherein the reverse primer additionally comprises a terminal 3′ cap.

19. The primer pair of any one of claims 16-18, wherein the non-natural nucleotide base of forward primer is the same as the non-natural nucleotide base of reverse primer.

20. The primer pair of any one of claims 16-18, wherein the non-natural nucleotide base of forward primer is different from the non-natural nucleotide base of reverse primer.

21. The primer pair of any one of claims 16-20, wherein the non-natural nucleotide base of forward primer is a ribonucleotide and the non-natural nucleotide base of reverse primer is a ribonucleotide.

22. The primer pair of claim 17, wherein the non-natural nucleotide base of forward primer is a ribonucleotide and the non-natural nucleotide base of reverse primer is a deoxyribonucleotide.

23. A method of detecting a target nucleic acid sequence, the method comprising:
a. providing a reaction mixture comprising
i. the primer pair of any one of claims 16-22;
ii. a sample nucleic acid that may or may not comprise the target sequence;
iii. a cleaving activity; and
iv. a polymerase activity;
b. hybridizing a first primer of the primer pair to the target nucleic acid sequence, if present, to form a first double-stranded substrate comprising the target nucleic acid sequence and a first target-specific cleavage domain;
c. cleaving the hybridized first primer with a first cleaving activity at a point within or adjacent to a first target-specific cleavage domain;
d. extending the primer with the polymerase activity to form a first template;
e. hybridizing a second primer of the primer pair to the first template to form a second double-stranded substrate comprising the target nucleic acid sequence and a second target-specific cleavage domain;
f. cleaving the hybridized second primer with a second cleaving activity at a point within or adjacent to the second cleavage domain;
g. extending the primer with the polymerase activity to form a first amplicon comprising, on a first strand, a first non-natural nucleotide base derived from the first primer;
h. cleaving the first strand at or adjacent to the first non-natural nucleotide with a third cleavage activity, creating a first nick with a 3′ hydroxyl group; and
i. extending, from the 3′ hydroxyl group, with the polymerase activity, displacing the portion of the first stand 3′ of the first nick, optionally wherein the first, second, and third cleavage activities are the same.

24. The method of claim 22, wherein terminal 3′ end of each primer comprises a terminal 3′ cap.
